(19) United States
(12) Patent Application Publication
(10) Pub. No.: US 2013/0136712 A1
(43) Pub. Date: May 30, 2013

(54) Malodor Control Compositions Comprising Malodor Control Polymers And Acid Catalysts And Methods Thereof

(71) Applicants: Ricky Ah-Man WOO, Hamilton, OH (US); Cahit Eylem, West Chester, OH (US); Larissa Azirbayeva, Mason, OH (US); Zaiyou Liu, West Chester, OH (US); Radhakrishnan Janardanan Nair, Kobe (JP); Kevin Robert Johnstone, Cincinnati, OH (US); Steven Anthony Horenziak, Cincinnati, OH (US); Michael-Vincent Nario Malanyaon, Indian Springs, OH (US); Rhonda Jean Jackson, Cincinnati, OH (US); Jason John Olchovy, West Chester, OH (US); Christine Marie Readnour, Fort Mitchell, KY (US)

(72) Inventors: Ricky Ah-Man WOO, Hamilton, OH (US); Cahit Eylem, West Chester, OH (US); Larissa Azirbayeva, Mason, OH (US); Zaiyou Liu, West Chester, OH (US); Radhakrishnan Janardanan Nair, Kobe (JP); Kevin Robert Johnstone, Cincinnati, OH (US); Steven Anthony Horenziak, Cincinnati, OH (US); Michael-Vincent Nario Malanyaon, Indian Springs, OH (US); Rhonda Jean Jackson, Cincinnati, OH (US); Jason John Olchovy, West Chester, OH (US); Christine Marie Readnour, Fort Mitchell, KY (US)

(21) Appl. No.: 13/305,778

(22) Filed: Nov. 29, 2011

(51) Int. Cl. A61L 9/01

(52) U.S. Cl. 424/76.1

(57) ABSTRACT

Compositions comprising a metallated malodor control polymer or a hydrophobically modified malodor control polymer, a malodor counteractant comprising a perfume material, and an acid catalyst; and methods thereof are provided. Such compositions may be used to reduce or neutralize malodors on surfaces or in the air.

### CROSS-REFERENCES TO RELATED APPLICATIONS

This application claims priority under 35 U.S.C. §120 to U.S. application Ser. No. 12/962,691 filed Dec. 8, 2010 and to U.S. Application Serial No. US2010/059618 filed Dec. 9, 2010 and U.S. application Ser. No. 13/006,644 filed Jan. 14, 2011.

### FIELD OF THE INVENTION

The present invention relates to compositions comprising malodor control polymers, a perfume mixture, and an acid catalyst, and methods thereof. The malodor control composition is suitable for use in a variety of applications, including use in fabric and air freshening products.

### BACKGROUND OF THE INVENTION

Products for reducing or masking malodors are currently available and are widely described in patent literature. These products may be designed to work specifically in air, on fabrics, or on other surfaces. However, not all malodors are effectively controlled by products in the market. Amine-based malodors such as fish and urine malodors and sulfur-based malodors such as garlic, onion, foot, and fecal malodors are difficult to combat. Further, the time required for a product to noticeably combat malodors may create consumer doubt as to a product's efficacy on malodors. For example, a consumer may leave the treated space before the product begins to noticeably reduce the malodors. Even further, certain compositions may cause fabrics on surrounding surfaces to turn yellow or brown under natural light and/or make fabrics susceptible to soiling, particularly compositions that contain certain types or amounts of aldehydes and/or surfactants. The difficulty in overcoming a broad range of malodors has spawned a diverse assortment of products to neutralize, mask, or contain malodors.

There remains a continuing need for a malodor control composition that neutralizes a broad range of malodors, including amine-based and sulfur-based malodors, while not overpowering malodors with an overwhelming perfume and while not soiling and staining fabrics.

### SUMMARY OF THE INVENTION

According to one embodiment of the present invention, there is provided a composition for reducing malodor comprising (a) an effective amount of a malodor control polymer selected from the group consisting of: (i) a metallated malodor control polymer comprising a water-soluble metal ion and a polymer selected from the group consisting of: partially hydrolyzed polyvinylamine (PVam), partially hydrolyzed hydrophobically modified PVam, polyethyleneimine (PEI), hydrophobically modified (PEI), polyamidoamine (PAMam), hydrophobically modified PAMam, polyallyamine (PAam), hydrophobically modified PAam, polyetheramine (PEam), hydrophobically modified PEam, and mixtures thereof; and (ii) a malodor control polymer having the structure (I):

P(R)x (I)

wherein P is selected from the group consisting of: partially hydrolyzed PVam, PEI, PAMam, PAam, PEam, and mixtures thereof; x is degree of substitution of the amine sites on the polymer and is less than 100%; and R is a C2 to C26 alkyl or alkenyl; and (iii) mixtures thereof; (b) a malodor counteractant comprising a perfume mixture comprising at least one volatile aldehyde; (c) an acid catalyst having a vapor pressure of about 0.01 to about 13 at 25° C.; and (d) an aqueous carrier; wherein said composition comprises a pH of about 5 to about 10.

According to another embodiment, there is provided a method of reducing malodor comprising the steps of: (1) providing a composition comprising (a) an effective amount of a malodor control polymer selected from the group consisting of: (i) a metallated malodor control polymer comprising a water-soluble metal ion and a polymer selected from the group consisting of: partially hydrolyzed PVam, partially hydrolyzed hydrophobically modified PVam, PEI, hydrophobically modified PEI, PAMam, hydrophobically modified PAMam, PAam, hydrophobically modified PAam, PEam, hydrophobically modified PEam, and mixtures thereof; (ii) a malodor control polymer having the structure (I):

P(R)x (I)

wherein P is selected from the group consisting of: partially hydrolyzed PVam, PEI, PAMam, PAam, PEam, and mixtures thereof; x is degree of substitution of the amine sites on the polymer and is less than 100%; and R is a C2 to C26 alkyl or alkenyl; and (iii) mixtures thereof; and (b) a malodor counteractant comprising a perfume mixture comprising at least one volatile aldehyde; (c) an acid catalyst having a vapor pressure of about 0.01 to about 13 at 25° C.; and (d) an aqueous carrier; wherein said composition comprises a pH of about 5 to about 10; and (b) dispersing an effective amount of said composition on an inanimate surface or in the air.

### BRIEF DESCRIPTION OF THE DRAWINGS

FIG. 1 is a graph showing the performance of a perfume mixture having 10 wt. % volatile aldehydes, in accordance with the present invention, on a sulfur-based malodor.

FIG. 2 is a graph showing the performance of a perfume mixture having 10 wt. % volatile aldehydes, in accordance with the present invention, on an amine-based malodor.

FIG. 3 is a graph showing butanethiol reduction by thiophene carboxaldehyde in combination with various acid catalysts.

### DETAILED DESCRIPTION OF THE INVENTION

The composition of the present invention is designed to deliver genuine malodor reduction and not function merely by using perfume to cover up or mask odors. A genuine malodor reduction provides a sensory and analytically measurable (e.g. gas chromatograph) malodor reduction. Malodors may include odors from food such as fish, onion, and garlic; odors from grease, body, mold/mildew, smoke, pet urine, sewage; and bathroom based odors. Thus, if the composition delivers a genuine malodor reduction, the composition will neutralize malodors in the air, on fabrics, and/or on other surfaces.

“Neutralize” or “neutralization” as used herein means chemically reacting with malodor components (e.g. the reaction of primary amines with aldehydes to form imines, reductive alkylation of amines, protonation and deprotonation of amines, polymerization or de-polymerization); or suppressing the volatility of malodorous components such that other parts of the composition may react (e.g. acid-base neutralization); or physically entrapping odorous molecules such that they are not re-released into the air (e.g. cyclodextrin inclusion complexes as described herein).

The composition may also act as a barrier to prevent malodors from adhering to or penetrating a surface.

### I. Malodor Control Composition

The malodor control composition comprises an effective amount of a malodor control polymer, a malodor counteractant comprising a perfume material, and an aqueous carrier.

In one embodiment, the composition may be free of ingredients that soil or stain fabrics treated with or surrounding the treated surface. In such embodiments, the total amount of surfactants (e.g. solubilizer, wetting agent) in the composition is from 0% to about 3% or no more than about 3%, alternatively from 0% to about 1% or no more than about 1%, alternatively from 0% to about 0.9% or no more than about 0.9%, alternatively from 0% to about 0.7% or no more than 0.7%, alternatively from 0% to about 0.5% or no more than about 0.5%, alternatively from 0% to about 0.3% or no more than about 0.3%, by weight of the composition. Compositions with higher concentrations may make fabrics susceptible to soiling and/or leave unacceptable visible stains on fabrics as the solution evaporates.

A. Hydrophobically Modified Malodor Control Polymers

The composition of the present invention includes a hydrophobically modified malodor control polymer (HMP). A HMP is formed from a polyamine polymer having a primary, secondary, and/or tertiary amine group that is modified with a hydrophobic group such as an alkyl, alkyloxide, or amide. Although the amine group has been modified, a HMP has at least one free and unmodified primary, secondary, and/or tertiary amine group, to react with malodorous components. Not wishing to be bound by theory, hydrophobic modification may increase a polymer's affinity for hydrophobic odors, thus enabling interactions between the odor molecules and active amine sites.

A HMP of the present invention has the general formula (I):

P(R)x (I)

wherein:

P is a polyamine polymer;

R is a C2 to C26 hydrophobic group; and

x is the total degree of substitution, which is less than 100%, of amine sites on the polymer.

1. Polyamine Polymer

HMPs may include a polyamine polymer backbone that can be either linear or cyclic. HMPs can also comprise polyamine branching chains to a greater or lesser degree. The polyamine polymer has a general formula (II):

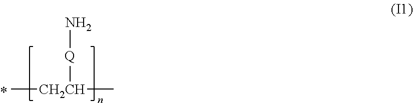

where Q is an integer having values between 0-3.

Non-limiting examples of polyamine polymers include polyvinylamines (PVams), polyethyleneimines (PEIs) that are linear or branched, polyamidoamines (PAMams), polyallyamines (PAams), polyetheramines (PEams) or other nitrogen containing polymers, such as lysine, or mixtures of these nitrogen containing polymers.

a. PVams

In one embodiment, the HMP includes a PVam backbone. A PVam is a linear polymer with pendent, primary amine groups directly linked to the main chain of alternating carbons. PVams are manufactured from hydrolysis of poly(N-vinylformamide) (PVNF) which results in the conversion of formamide units to amino groups as described by the following formula (IIa):

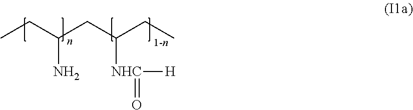

where n is a number from 0.1 to 0.99 depending on the degree of hydrolysis. For instance, in 95% hydrolyzed PVam polymer, n will be 0.95 while 5% of the polymer will have formamide units.

PVams may be partially hydrolyzed meaning that 1% to 99%, alternatively 30% to 99%, alternatively 50% to 99%, alternatively 70% to 99%, alternatively 80% to 99%, alternatively 85% to 99%, alternatively 90% to 99%, alternatively 95% to 99%, alternatively 97% to 99%, alternatively 99% of the PVam is hydrolyzed. It has been found that high degree of hydrolysis of PVam increases the resulting polymer's ability to mitigate the odors.

PVams that can be hydrolyzed may have an average molecular weight (MW) of 5,000 to 350,000. Suitable hydrolyzed PVams are commercially available from BASF. Some examples include Lupamin™ 9095, 9030, 5095, and 1595.

Such hydrolyzed PVams may then be hydrophobically modified. Hydrophobic modification, as described herein, may further improve malodor removal efficacy.

b. Polyalkylenimine/PEIs

In another embodiment, the HMP includes a polyalkylenimine backbone. Polyalkylenimines include PEIs and polypropylenimines as well as the C4-C12 alkylenimines.

PEI is a suitable polyalkylenimine. The chemical structure of a PEI follows a simple principle: one amine function and two carbons. PEIs have the following general formula (IIb):

—(CH2-CH2-NH)n- (IIb):

where n=10-105

PEIs constitute a large family of water-soluble polyamines of varying molecular weight, structure, and degree of modification. They may act as weak bases and may exhibit a cationic character depending on the extent of protonation driven by pH.

PEIs are produced by the ring-opening cationic polymerization of ethyleneimine as shown below.

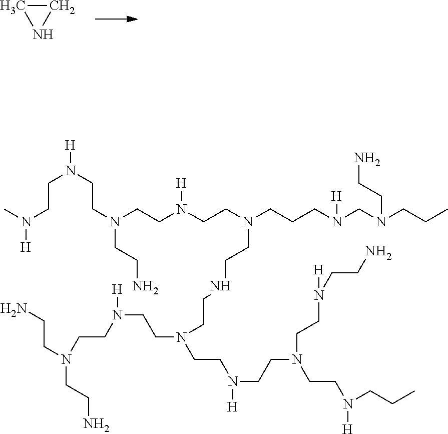

PEIs are believed to be highly branched containing primary, secondary, and tertiary amine groups in the ratio of about 1:2:1. PEIs may comprise a primary amine range from about 30% to about 40%, alternatively from about 32% to about 38%, alternatively from about 34% to about 36%. PEIs may comprise a secondary amine range from about 30% to about 40%, alternatively from about 32% to about 38%, alternatively from about 34% to about 36%. PEIs may comprise a tertiary amine range from about 25% to about 35%, alternatively from about 27% to about 33%, alternatively from about 29% to about 31%.

Other routes of synthesis may lead to products with a modified branched chain structure or even to linear chain PEIs. Linear PEIs contain amine sites in the main chain while the branched PEIs contain amines on the main and side chains. Below is an example of a linear PEI

### Linear PEI

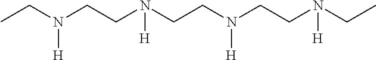

The composition of the present invention may comprise PEIs having a MW of about 800 to about 2,000,000, alternatively about 1,000 to about 2,000,000, alternatively about 1,200 to about 25,000, alternatively about 1,300 to about 25,000, alternatively about 2,000 to about 25,000, alternatively about 10,000 to about 2,000,000, alternatively about 25,000 to about 2,000,000, alternatively about 25,000.

In one embodiment, the PEI may have a specific gravity of 1.05 and/or an amine value of 18 (mmol/g, solid). For clarity, such specific gravity and/or amine value of the PEI describes the PEI before it is modified or added as part of an aqueous composition. One skilled in the art will appreciate, for example, the primary and secondary amino groups may react with other components of the composition.

Exemplary PEIs include those that are commercially available under the tradename Lupasol® from BASF or the tradename Epomine™ from Nippon Shokubia.

In some embodiments, less than 100% of the active amine sites are substituted with hydrophobic functional groups, alternatively about 0.5% to about 90%, alternatively about 0.5% to about 80%, alternatively about 0.5% to about 70%, alternatively about 0.5% to about 60%, alternatively about 0.5% to about 50%, alternatively about 0.5% to about 40%, alternatively about 0.5% to about 35%, alternatively about 0.5% to about 30%, alternatively about 1% to about 30%, alternatively about alternatively about 1% to about 25%, alternatively about 1% to about 20%, alternatively about 5% to about 20%, alternatively about 10% to about 30%, alternatively about 20% to about 30%, alternatively about 20% of the active amine sites are substituted with hydrophobic functional groups. When a PEI has active amine sites that are fully substituted with hydrophobic functional groups, such hydrophobically modified PEI may have no activity for malodor control.

c. PAMams

In another embodiment, the HMP includes a PAMam backbone. PAMams are polymers whose backbone chain contains both amino functionalities (NH) and amide functionalities (NH—C(O)). PAMams also contain primary amine groups and/or carboxyl groups at the termini of polymer chain. The general structure of a PAMam is below (IIc):

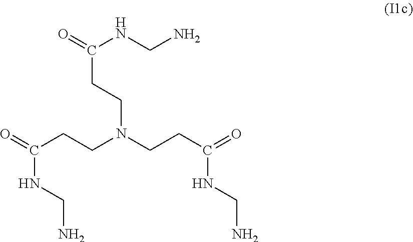 [SMILES: NCNC(=O)CCN(CCC(=O)NCN)CCC(=O)NCN]

d. PAams

In another embodiment, the HMP includes a PAam backbone. PAams are prepared from polymerization of allyamine —C₃H₅NH2. Unlike PEIs, they contain only primary amino groups that are linked to the side chains. The general formula for a PAam is shown below (II):

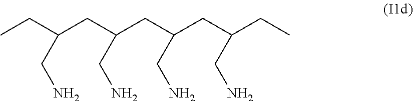

e. PEams

In yet another embodiment, the HMP includes a PEam backbone. PEams contain a primary amino groups attached to the end of a polyether backbone. The polyether backbone may be based on propylene oxide (PO), ethylene oxide (EO), or mixed PO/EO. The general formula for a PEam is shown below (II):

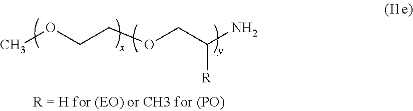

These so-called monoamines, M-series, are commercially available from Hunstman under the tradename Jeffamine® monoamines. In another embodiment, the HMP includes a PEam backbone having diamines as shown below (IIf):

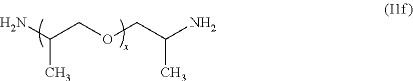

Diamines are commercially available from Hunstman under the tradename Jeffamine® diamines (e.g. D, ED, and EDR series). The HMP may also include a PEam backbone having triamines (e.g. Jeffamine® triamine T-series).

2. Other Polymer Units

HMPs may include a copolymer of nitrogen-containing polymers having the formula (I2):

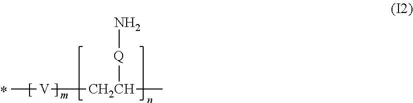

where Q is an integer having values between 0-3 and V is a co-monomer.

Non-limiting examples of (I2) unmodified polymers include vinylamides, vinyl pyrrolidone, vinylimidazole, vinylesters, vinylalcohols, and mixtures thereof.

3. Hydrophobic Group

The hydrophobic group of the HMP may be linear, branched, or cyclic alkyl, hydroxyalkyl, alkenyl, hydroxyalkenyl, alkyl carboxyl, alkyloxide, alkanediyl, amide, or aryl. In some embodiments, the hydrophobic group is a C2 to C26, alternatively a C2 to C12, alternatively a C2 to C10, alternatively a C4 to C10, alternatively a C16 to C26, alternatively a C6. Where cyclodextrin is included in a formulation, it may be desirous to use a HMP that has been modified with a C2 to C10 alkyl group, alternatively a C16-C26 alkyl group, alternatively a C6 alkyl group, since such alkyl groups are cyclodextrin compatible.

4. Hydrophobic Modification

The polyamine backbones are hydrophobically modified in such a manner that at least one nitrogen, alternatively each nitrogen, of the polyamine chain is thereafter described in terms of a unit that is substituted, quaternized, oxidized, or combinations thereof.

There are many ways of hydrophobically modifying polyamine polymers. Generally, the modification is one directed to the primary, secondary, and/or tertiary amines of the polymer. By reacting the unmodified polyamine backbone with appropriate reagents, one can render the polyamine polymer hydrophobic, thereby increasing efficacy for malodor removal. The following are non limiting examples of the ways to prepare the HMPs disclosed herein.

a. Alkoxylation

The reaction of polyamine polymer with an epoxide containing hydrocarbons (R) results in substitution of one or more nitrogen moities on the polymer.

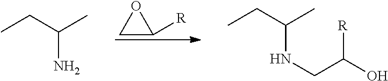

wherein R>C2.

Non-limiting example of such hydrocarbons include C2-C26 chain that is substituted or unsubstituted, branched or unbranched. For example, a reaction of dodeceneoxide with PEI polymer results in C6-HMP disclosed herein having a structure shown below.

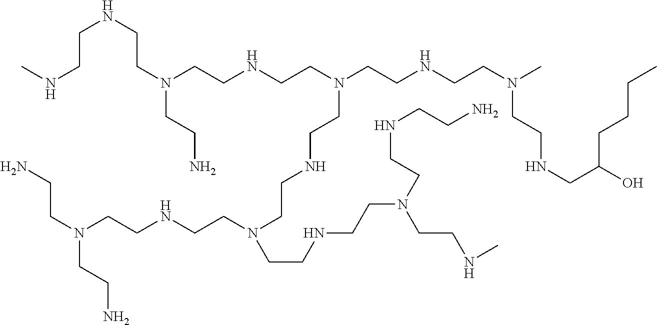 [SMILES: CCCCC(O)CNCCN(C)CCNCCN(CCNCCN(CCN)CCNCCNC)CCNCCN(CCNCCN(CCN)CCN)CCNCCN(CCNC)CCNCCN]

Alternatively, one can modify the base polymer by reacting with EO first and then finish it by alkylation. Additional modifications might also include capping the modified polymer with EO groups if more water solubility is desired. Alternatively, hydroxyl groups can be substituted by further reacting the alkoxylated polymers as described in subparagraph c below.

b. Amidation

Reaction of polyamine polymers with amide-forming reagents such as anhydrides, lactones, isocyanates, or carboxylic acids results in substitution of one or more nitrogen moieties on the polymer rendering hydrophobic character. Prior to amidation, one can begin with partial substitution of amine sites with EO or PO and then carry out amidation on the remaining amine moieties. Reaction of anhydrides with polyamine polymers leads to the formation of amide units of the polymer by partial substitution of the primary/secondary amine sites. Non-limiting examples include non-cyclic carboxylic anhydrides such as acetic anhydride or cyclic carboxylic anhydrides such as maleic anhydride, succinic anhydride or phthalic anhydride. For example, the reaction of a polyamine with acetic anhydride introduces amide units onto the polymer.

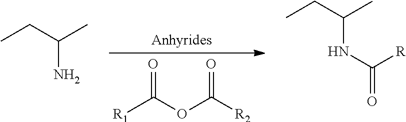

wherein R>C2.

On the other hand, the reaction of polyamine polymer with cyclic anhydrides introduces amido acid units onto the polymer.

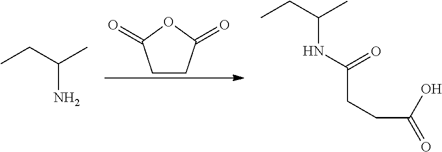

More hydrophobically modified derivatives can be prepared by the use of cyclic anhydrides such as alkylene succinic anhydrides, dodecenyl succinic anhydride, or polyisobutane succinic anhydride.

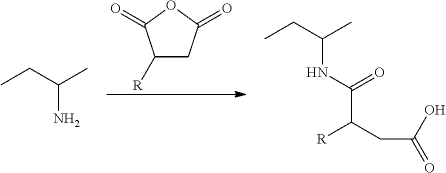

wherein R>C2.

Polyamine polymers containing hydroxyl-terminated polyamido units can be prepared by reacting the polymers with lactones. The use of more hydrophobic alkyl substituted lactones may introduce more hydrophobicity. Optionally, hydroxyl-end groups can be further substituted with functional groups as described in the following subparagraph c.

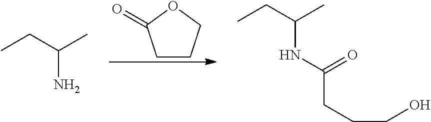

Isocyanate reactions with polyamine polymers result in the formation of urea derivatives as shown below.

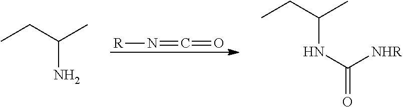

wherein R>C2.

c. Alkoxylation Followed by Substitution of Hydroxyl Groups

Additional functional groups can be covalently bonded to an OH group on the alkoxylated polyamine polymers (“x” in formula (I)). This can be achieved by further reacting the alkoxylated polymers with bifunctional compounds such as epihalohydrins such as epichlorohydrin, 2-halo acid halides, isocyanataes or disocyanates such as trimethylhexane diisocyanate, or cyclic carboxylic anhydrides such as maleic anhydride or phthalic anhydride. For example, the reaction of alkoxylated PEI with isocyanates yields:

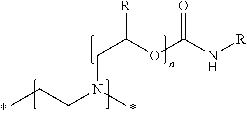

wherein R>C2.

Reaction products of alkoxylated PEI and alk(en)ylsuccinic anhydrides yield

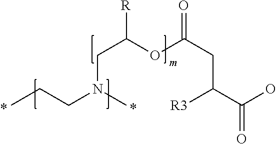

wherein R>C2.

All these HMPs disclosed herein can be optionally capped with hydrophilic groups, such as EO, to render water solubility if necessary.

In some embodiments, about 0.5% to about 90% of the amine groups on the entire unmodified polyamine polymer may be substituted with a hydrophobic group, alternatively about 0.5% to about 80%, alternatively about 0.5% to about 70%, alternatively about 0.5% to about 60%, alternatively about 0.5% to about 50%, alternatively about 0.5% to about 40%, alternatively about 0.5% to about 35%, alternatively about 0.5% to about 30%, alternatively about 1% to about 30%, alternatively about alternatively about 1% to about 25%, alternatively about 1% to about 20%, alternatively about 5% to about 20%, alternatively about 10% to about 30%, alternatively about 20% to about 30%, alternatively about 20% of the amine groups on the entire unmodified polyamine polymer may be substituted with a hydrophobic group. The level of substitution of the amine units can be as low as 0.01 mol percent of the theoretical maximum where all primary, secondary, and/or tertiary amine units have been replaced.

HMPs for use herein may have a MW from about 150 to about 2* 10⁶, alternatively from about 400 to about 10⁶, alternatively from about 5000 to about 10⁶.

Malodor control polymers suitable for use in the present invention are water-soluble or dispersible. In some embodiments, the primary, secondary, and/or tertiary amines of the polyamine chain are partially substituted rendering hydrophobicity while maintaining the desired water solubility. The minimum solubility index of a HMP may be about 2% (i.e. 2g/100 ml of water). A suitable HMP for an aqueous fabric refresher formulation may have a water solubility percentage of greater than about 0.5% to 100%, alternatively greater than about 5%, alternatively greater than about 10%, alternatively greater than about 20%.

The water solubility index can be determined by the following test.

### Water Solubility

This test illustrates the benchmarking ambient temperature water solubility of HMPs against beta-cyclodextrin (1.8 g/100 ml) and hydroxypropyl modified beta cyclodextrin (60+g/100 ml). 1% water solubility is used as a screening criteria for HMPs suitable for use in aqueous fabric refresher formulations.

Room temperature equilibrium water solubility of polymers may be determined by adding weighed quantities of polymers into 100 ml of deionized water and allowing the added polymers to completely dissolve. This process is repeated until the added polymers are no longer soluble. Equilibrium water solubility is then calculated based on how much polymer is dissolved in 100 ml water.

| | Equilibrium Water Solubility |
|---|---|
| Polymer | (g/100 ml water at 25° C.) |
| Lupasol G100 (PEI 5,000) | miscible at all levels (70+) |
| C6 modified PEI 1800 | 30+ |
| (0.25 C6/NH) | |
| Dodecene oxide modified PEI5,000 | ~24 |
| (0.1 dodecene oxide/NH) | |
| Dodecene oxide modified PEI5,000 | ~4 |
| (0.2 dodecene oxide/NH) | |
| Dodecene oxide modified PEI5,000 | <0.1 |
| (0.5 dodecene oxide/NH) | |
| Dodecene oxide modified PEI25,000 | ~21 |
| (0.1 dodecene oxide/NH) | |
| Dodecene oxide modified PEI25,000 | <0.1 |
| (0.2 dodecene oxide/NH) | |
| Dodecene oxide and EO modified | ~6 |
| PEI25,000 (0.8 EO and 0.2 dodecene | |
| oxide/NH) | |

When the polymer is not water soluble (e.g. less than 0.05%), capping with a hydrophilic molecule may be desired to assist with water solubility. Suitable hydrophilic molecules include EO or other suitable hydrophilic functional groups.

Suitable levels of HMPS in the present composition are from about 0.01% to about 10%, alternatively from about 0.01% to about 2%, alternatively from about 0.01% to about 1%, alternatively from about 0.01% to about 0.8%, alternatively from about 0.01% to about 0.6%, alternatively from about 0.01% to about 0.1%, alternatively from about 0.01% to about 0.07%, alternatively about 0.07%, alternatively about 0.5%, by weight of the composition. Compositions with higher amount of HMPs may make fabrics susceptible to soiling and/or leave unacceptable visible stains on fabrics as the composition evaporates off of the fabric.

Suitable HMPs incude partially hydrolyzed hydrophobically modified PVams, hydrophobically modified PEIs, hydrophobically modified PAMams, hydrophobically modified PAams, and mixtures thereof.

B. Metal Coordinated Complexes

The malodor control composition of the present invention may include a malodor control polymer that is a metal coordinated complex or a metallated polymer. The metal coordinated complex comprises a metal and any unmodified polymer disclosed herein (i.e. polyamine polymers), a HMP disclosed herein, or mixtures thereof. Metal coordination may improve the odor neutralization of a malodor control polymer. Metal coordination might also provide reduction of malodor from microbial sources. Suitable metals that coordinate with such polymers include zinc, copper, silver, and mixtures thereof. Suitable metals also include Na, K, Ca, Mg, and non-transition metals, including Sn, Bi, and Al.

Metals that are not coordinated to a polymer may deliver some malodor control using highly ionizable water soluble salts such as zinc chloride or silver nitrate. But, such metals present drawbacks in aqueous formulations. Zinc ions and silver ions have the ability to form insoluble salts with nucleophilic compounds such as valeric acid, skatole, hydrogen sulfide, mercaptan, and like compounds that are typically the cause of environmental malodor. However, zinc chloride aqueous solutions, over time, tend to form insoluble oxychlorides and hydroxides that have low water solubility. As a result, aqueous formulations containing zinc chloride are traditionally kept below pH 4.5 in order to avoid the formation of these insoluble salts that result in cloudy formulations. Just like zinc salts, silver compounds suffer from pH stability, formation of insoluble salts with anions typically present in water. Silver ion, additionally, is very light sensitive and can easily be first reduced to silver metal by photo-reduction process and then oxidized to black silver oxide after lengthy light exposure. For aqueous spray applications, these issues may be considered detriments.

Coordinating zinc ion or silver ion with polyamine polymers may overcome the limitations described above, resulting in water soluble complexes with a wide range pH stability (e.g. >4.5). Additionally, these complexes may provide synergistic malodor control and prevention efficacy not previously seen with the polymers and metal salts, such as zinc chloride, alone. For example, by coordinating zinc ions with HMPs, we also discovered efficacy for hydrophobic sulfur odors which traditional zinc salts are not effective against. Because hydrophobic modification might decrease Zn binding capacity as well as water solubility of the polymer, one may wish to control the degree of such modification.

Nitrogen containing polymers, such as PEIs, have high binding capacity for metals due to availability of basic nitrogen sites. The strength of the metal-nitrogen ligand interaction is influenced by several factors including the microstructure of polymer, functionality of the binding sites, the density of nitrogen ligands in the polymer, steric constraints, electrostatic interactions, pH, pKa of the polymer, and oxidation state, size, and electronic configuration of metal. Unlike traditional chelators, such as ethylenediamine (bi-dentate), ethylenediaminetetraacetic acid, or EDTA (hexa-dentate), polymers can be considered poly-dentate due to high density of binding sites. As a result, the chemical formula of metal coordination complex is highly variable.

In some embodiments, the metal coordinated complex is a HMP having at least 5% of its primary, secondary, and/or tertiary amine sites left unmodified for not only malodor efficacy but also for metal binding capacity.

Metal coordinated complexes may have a metal / polymer weight ratio from 0.001 and 50, alternatively from 0.001 to 20, alternatively from 0.001 to 15, alternatively from 0.001 to 10, alternatively from 0.005 to 5.0, alternatively from 0.1 to 1.0, alternatively from 0.1 to 0.5, alternatively from 0.001 to 0.01.

Metal polymer coordination complexes can be prepared by reacting suitable metal salts with polyamine polymers containing primary or secondary amine sites. The resulting complex can be represented by a general formula, MxPy; where M is metal, P is an unmodified polyamine polymer or a HMP, and x and y are integers and dependent on coordination number of metal ion, number of available coordinating sites on the polymer, and pH.

It is believed there is strong competition between the metal ions and protons for the electron pairs on the amine groups of polyamine polymers. This competition is favored for the metal ions at higher pH values, where amine groups are deprotonated and more available for metal binding. It may be assumed that only the non-protonated nitrogen sites of the polymers are active towards the metal ions; then polyamine polymers will have the highest metal binding capacity at high pH levels. Metal ions can coordinate to four to eight ligands. Zinc ion is known to prefer 4-coordinated tetrahedral sites as shown below, while copper ions tend to form octahedral coordinations. Examples of possible zinc polymer structures are shown below. For example, zinc ion can bind to 2 nitrogen units on each PVam. Alternatively, a polymer can fold around zinc ion and utilize four nitrogen to form tetrahedral coordination.

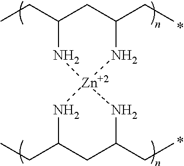

Protonation and metal binding ability of polyamine polymers are also influenced by polymer microstructure. For instance, branched PEIs have amine sites located in the main and side chains whereas PVams have only primary amino groups linked directly to the main chain.

As a result, PVams having ligands only in the side chain are of greater advantage for protonation than the case having in the main chain branched PEIs. Therefore, one might expect different metal binding capacities for PVam and PEI at the same pH levels. Due to its linear structure, PVams show relatively strong interaction in neighboring ammonium groups on the polymer chain in comparison to branched PEIs. This difference is also expected to influence the metal binding capacity of the polymers.

In one embodiment, the composition is includes a zinc polymer complex having a pH of 7. It is believed that at such pH the competition between protonation and metal coordination of amine sites provides a unique coordination environment for zinc. This unique bonding makes the zinc ions readily available for additional interactions with malodor molecules, while preventing the release of zinc ions from the metal coordinated complex.

C. Malodor Counteractants

The composition may utilize one or more malodor counteractants. Malodor counteractants may include components which lower the vapor pressure of odorous compounds, solubilize malodor compounds, physically entrap odors (e.g. flocculate or encapsulate), physically bind odors, or physically repel odors from binding to inanimate surfaces.

1. Perfume Mixture Comprising At Least One Volatile Aldehyde

The malodor control composition includes a perfume mixture comprising at least one volatile aldehyde that neutralizes malodors in vapor and/or liquid phase via chemical reactions. Such volatile aldehydes are also called reactive aldehydes (RA). Volatile aldehydes may react with amine-based odors, following the path of Schiff-base formation. Volatiles aldehydes may also react with sulfur-based odors, forming thiol acetals, hemi thiolacetals, and thiol esters in vapor and/or liquid phase. It may be desirable for these vapor and/or liquid phase volatile aldehydes to have virtually no negative impact on the desired perfume character of a product. Aldehydes that are partially volatile may be considered a volatile aldehyde as used herein.

Suitable volatile aldehydes may have a vapor pressure (VP) in the range of about 0.0001 torr to 100 torr, alternatively about 0.0001 torr to about 10 torr, alternatively about 0.001 torr to about 50 torr, alternatively about 0.001 torr to about 20 torr, alternatively about 0.001 torr to about 0.100 torr, alternatively about 0.001 torr to 0.06 torr, alternatively about 0.001 torr to 0.03 torr, alternatively about 0.005 torr to about 20 torr, alternatively about 0.01 torr to about 20 torr, alternatively about 0.01 torr to about 15 torr, alternatively about 0.01 torr to about 10 torr, alternatively about 0.05 torr to about 10 torr, measured at 25° C.

The volatile aldehydes may also have a certain boiling point (B.P.) and octanol/water partition coefficient (P). The boiling point referred to herein is measured under normal standard pressure of 760 mmHg. The boiling points of many volatile aldehydes, at standard 760 mm Hg are given in, for example, “Perfume and Flavor Chemicals (Aroma Chemicals),” written and published by Steffen Arctander, 1969.

The octanol/water partition coefficient of a volatile aldehyde is the ratio between its equilibrium concentrations in octanol and in water. The partition coefficients of the volatile aldehydes used in the malodor control composition may be more conveniently given in the form of their logarithm to the base 10, logP. The logP values of many volatile aldehydes have been reported. See, e.g., the Pomona92 database, available from Daylight Chemical Information Systems, Inc. (Daylight CIS), Irvine, California. However, the logP values are most conveniently calculated by the “CLOGP” program, also available from Daylight CIS. This program also lists experimental logP values when they are available in the Pomona92 database. The “calculated logP” (ClogP) is determined by the fragment approach of Hansch and Leo (cf., A. Leo, in Comprehensive Medicinal Chemistry, Vol. 4, C. Hansch, P. G. Sammens, J. B. Taylor and C. A. Ramsden, Eds., p. 295, Pergamon Press, 1990). The fragment approach is based on the chemical structure of each volatile aldehyde, and takes into account the numbers and types of atoms, the atom connectivity, and chemical bonding. The ClogP values, which are the most reliable and widely used estimates for this physicochemical property, are preferably used instead of the experimental logP values in the selection of volatile aldehydes for the malodor control composition.

The ClogP values may be defined by four groups and the volatile aldehydes may be selected from one or more of these groups. The first group comprises volatile aldehydes that have a B.P. of about 250 ° C. or less and ClogP of about 3 or less. The second group comprises volatile aldehydes that have a B.P. of 250° C. or less and ClogP of 3.0 or more. The third group comprises volatile aldehydes that have a B.P. of 250° C. or more and ClogP of 3.0 or less. The fourth group comprises volatile aldehydes that have a B.P. of 250° C. or more and ClogP of 3.0 or more. The malodor control composition may comprise any combination of volatile aldehydes from one or more of the ClogP groups.

In some embodiments, the volatile aldehydes may comprise, by total weight of the perfume mixture, from about 0% to about 30% of volatile aldehydes from group 1, alternatively about 25%; and/or about 0% to about 10% of volatile aldehydes from group 2, alternatively about 10%; and/or from about 10% to about 30% of volatile aldehydes from group 3, alternatively about 30%; and/or from about 35% to about 60% of volatile aldehydes from group 4, alternatively about 35%.

Exemplary volatile aldehydes which may be used in a malodor control composition include, but are not limited to, Adoxal (2,6,10-Trimethyl-9-undecenal), Bourgeonal (4-t-butylbenzenepropionaldehyde), Lilestralis 33 (2-methyl-4-t-butylphenyl)propanal), Cinnamic aldehyde, cinnamaldehyde (phenyl propenal, 3-phenyl-2-propenal), Citral, Geranial, Neral (dimethyloctadienal, 3,7-dimethyl-2,6-octadien-1-al), Cyclal C (2,4-dimethyl-3-cyclohexen-1-carbaldehyde), Florhydral (3-(3-Isopropyl-phenyl)-butyraldehyde), Citronellal (3,7-dimethyl 6-octenal), Cymal, cyclamen aldehyde, Cyclosal, Lime aldehyde (Alpha-methyl-p-isopropyl phenyl propyl aldehyde), Methyl Nonyl Acetaldehyde, aldehyde C12 MNA (2-methyl-1-undecanal), Hydroxycitronellal, citronellal hydrate (7-hydroxy-3,7-dimethyl octan-1-al), Helional (alpha-methyl-3,4-(methylenedioxy)-hydrocinnamaldehyde, hydrocinnamaldehyde (3-phenylpropanal, 3-phenylpropionaldehyde), Intreleven aldehyde (undec-10-en-1-al), Ligustral, Trivertal (2,4-dimethyl-3-cyclohexene-1-carboxaldehyde), Jasmorange, satinaldehyde (2-methyl-3-tolylproionaldehyde, 4-dimethylbenzenepropanal), Lyral (4-(4-hydroxy-4-methyl pentyl)-3-cyclohexene-1-carboxaldehyde), Melonal (2,6-Dimethyl-5-Heptenal), Methoxy Melonal (6-methoxy-2,6-dimethylheptanal), methoxycinnamaldehyde (trans-4-methoxycinnamaldehyde), Myrac aldehyde isohexenyl cyclohexenyl-carboxaldehyde, trifernal ((3-methyl-4-phenyl propanal, 3-phenyl butanal), lilial, P.T. Bucinal, lysmeral, benzenepropanal (4-tert-butyl-alpha-methyl-hydrocinnamaldehyde), Dupical, tricyclodecylidenebutanal (4-Tricyclo5210-2,6decylidene-8butanal), Melafleur (1,2,3,4,5,6,7,8-octahydro-8,8-dimethyl-2-naphthaldehyde), Methyl Octyl Acetaldehyde, aldehyde C-11 MOA (2-mehtyl deca-1-al), Onicidal (2,6,10-trimethyl-5,9-undecadien-1-al), Citronellyl oxyacetaldehyde, Muguet aldehyde 50 (3,7-dimethyl-6-octenyl) oxyacetaldehyde), phenylacetaldehyde, Mefranal (3-methyl-5-phenyl pentanal), Triplal, Vertocitral dimethyl tetrahydrobenzene aldehyde (2,4-dimethyl-3-cyclohexene-1-carboxaldehyde), 2-phenylproprionaldehyde, Hydrotropaldehyde, Canthoxal, anisylpropanal 4-methoxy-alpha-methyl benzenepropanal (2-anisylidene propanal), Cylcemone A (1,2,3,4,5,6,7,8-octahydro-8,8-dimethyl-2-naphthaldehyde), and Precylcemone B (1-cyclohexene-1-carboxaldehyde).

Still other exemplary aldehydes include, but are not limited to, acetaldehyde (ethanal), pentanal, valeraldehyde, amylaldehyde, Scentenal (octahydro-5-methoxy-4,7-Methano-1H-indene-2-carboxaldehyde), propionaldehyde (propanal), Cyclocitral, beta-cyclocitral, (2,6,6-trimethyl-1-cyclohexene-1-acetaldehyde), Iso Cyclocitral (2,4,6-trimethyl-3-cyclohexene-1-carboxaldehyde), isobutyraldehyde, butyraldehyde, isovaleraldehyde (3-methyl butyraldehyde), methylbutyraldehyde (2-methyl butyraldehyde, 2-methyl butanal), Dihydrocitronellal (3,7-dimethyl octan-1-al), 2-Ethylbutyraldehyde, 3-Methyl-2-butenal, 2-Methylpentanal, 2-Methyl Valeraldehyde, Hexenal (2-hexenal, trans-2-hexenal), Heptanal, Octanal, Nonanal, Decanal, Laurie aldehyde, Tridecanal, 2-Dodecanal, Methylthiobutanal, Glutaraldehyde, Pentanedial, Glutaric aldehyde, Heptenal, cis or trans-Heptenal, Undecenal (2-, 10-), 2,4-octadienal, Nonenal (2-, 6-), Decenal (2-, 4-), 2,4-hexadienal, 2,4-Decadienal, 2,6-Nonadienal, Octenal, 2,6-dimethyl 5-heptenal, 2-isopropyl-5-methyl-2-hexenal, Trifernal, beta methyl Benzenepropanal, 2,6,6-Trimethyl-1-cyclohexene-1-acetaldehyde, phenyl Butenal (2-phenyl 2-butenal), 2.Methyl-3(p-isopropylphenyl)-propionaldehyde, 3-(p-isopropylphenyl)-propionaldehyde, p-Tolylacetaldehyde (4-methylphenylacetaldehyde), Anisaldehyde (p-methoxybenzene aldehyde), Benzaldehyde, Vernaldehyde (1-Methyl-4-(4-methylpentyl)-3-cyclohexenecarbaldehyde), Heliotropin (piperonal) 3,4-Methylene dioxy benzaldehyde, alpha-Amylcinnamic aldehyde, 2-pentyl-3-phenylpropenoic aldehyde, Vanillin (4-methoxy 3-hydroxy benzaldehyde), Ethyl vanillin (3-ethoxy 4-hydroxybenzaldehyde), Hexyl Cinnamic aldehyde, Jasmonal H (alpha-n-hexyl-cinnameldehyde), Floralozone, (para-ethyl-alpha,alpha-dimethyl Hydrocinnamaldehyde), Acalea (p-methyl-alpha-pentylcinnamaldehyde), methylcinnamaldehyde, alpha-Methylcinnamaldehyde (2-methyl 3-pheny propenal), alpha-hexylcinnamaldehyde (2-hexyl 3-phenyl propenal), Salicylaldehyde (2-hydroxy benzaldehyde), 4-ethyl benzaldehyde, Cuminaldehyde (4-isopropyl benzaldehyde), Ethoxybenzaldehyde, 2,4-dimethylbenzaldehyde, Veratraldehyde (3,4-dimethoxybenzaldehyde), Syringaldehyde (3,5-di methoxy 4-hydroxybenzaldehyde), Catechaldehyde (3,4-dihydroxybenzaldehyde), Safranal (2,6,6-trimethyl-1,3-diene methanal), Myrtenal (pin-2-ene-1-carbaldehyde), Perillaldehyde L-4(1-methylethenyl)-1-cyclohexene-1-carboxaldehyde), 2,4-Dimethyl-3-cyclohexene carboxaldehyde, 2-Methyl-2-pentenal, 2-methylpentenal, pyruvaldehyde, formyl Tricyclodecan, Mandarin aldehyde, Cyclemax, Pino acetaldehyde, Corps Iris, Maceal, and Corps 4322.

In one embodiment, the perfume mixture includes two or more volatile aldehydes selected from the group consisting of 2-ethoxy Benzylaldehyde, 2-isopropyl-5-methyl-2-hexenal, 5-methyl Furfural, 5-methyl-thiophene-carboxaldehyde, Adoxal, p-anisaldehyde, Benzylaldehyde, Bourgenal, Cinnamic aldehyde, Cymal, Decyl aldehyde, Floral super, Florhydral, Helional, Lauric aldehyde, Ligustral, Lyral, Melonal, o-anisaldehyde, Pino acetaldehyde, P.T. Bucinal, Thiophene carboxaldehyde, trans-4-Decenal, trans trans 2,4-Nonadienal, Undecyl aldehyde, and mixtures thereof.

In some embodiments, the perfume mixture includes fast reacting volatile aldehydes. “Fast reacting volatile aldehydes” refers to volatile aldehydes that either (1) reduce amine odors by 20% or more in less than 40 seconds; or (2) reduce thiol odors by 20% or more in less than 30 minutes.

In one embodiment, the perfume mixture includes the volatile aldehydes listed in Table 1 and referred to herein as Accord A.

TABLE 1
Accord A

| | Wt. % | | | |
|---|---|---|---|---|
| | (by weight of | | | |
| | the perfume | CAS | ClogP | VP(torr) |
| Material | mixture) | Number | Group | @25° C. |
| Intreleven Aldehyde | 5.000 | 112-45-8 | 3 | 0.060 |
| Florhydral | 10.000 | 125109-85-5 | 4 | 0.008 |
| Floral Super | 25.000 | 71077-31-1 | 3 | 0.030 |
| Scentenal | 10.000 | 86803-90-9 | 2 | 0.010 |
| Cymal | 25.000 | 103-95-7 | 4 | 0.007 |
| o-anisaldehyde | 25.000 | 135-02-4 | 1 | 0.032 |

In another embodiment, the perfume mixture includes the volatile aldehydes listed in Table 2 and referred to herein as Accord B.

TABLE 2
Accord B

| | Wt. % | | | |
|---|---|---|---|---|
| | (by weight of | | | |
| | the perfume | CAS | ClogP | VP (torr) |
| Material | mixture) | Number | Group | @25° C. |
| Intreleven Aldehyde | 2.000 | 112-45-8 | 3 | 0.060 |
| Florhydral | 20.000 | 125109-85-5 | 4 | 0.008 |
| Floral Super | 10.000 | 71077-31-1 | 3 | 0.030 |
| Scentenal | 5.000 | 86803-90-9 | 2 | 0.010 |
| Cymal | 25.000 | 103-95-7 | 4 | 0.007 |
| Floralozone | 10.000 | 67634-14-4 | 4 | 0.005 |
| Adoxal | 1.000 | 141-13-9 | 4 | 0.007 |
| Methyl Nonyl | 1.000 | 110-41-8 | 3 | 0.030 |
| Acetaldehyde | | | | |
| Melonal | 1.000 | 106-72-9 | 3 | 0.670 |
| o-anisaldehyde | 25.000 | 135-02-4 | 1 | 0.032 |

In another embodiment, the perfume mixture includes about 71.2% volatile aldehydes, the remainder being an ester and an alcohol perfume raw material. This mixture is listed in Table 3 and referred to herein as Accord C.

TABLE 3
Accord C

| | Wt. % | | | |
|---|---|---|---|---|
| | (by weight of | | | |
| | the perfume | CAS | ClogP | VP (torr) |
| Material | mixture) | Number | Group | @25° C. |
| Intreleven Aldehyde | 2.000 | 112-45-8 | 3 | 0.060 |
| Florhydral | 10.000 | 125109-85-5 | 4 | 0.008 |
| Floral Super | 5.000 | 71077-31-1 | 3 | 0.030 |
| Scentenal | 2.000 | 86803-90-9 | 2 | 0.010 |
| Cymal | 15.000 | 103-95-7 | 4 | 0.007 |
| Floralozone | 12.000 | 67634-14-4 | 4 | 0.005 |
| Adoxal | 1.000 | 141-13-9 | 4 | 0.007 |
| Methyl Nonyl | 1.000 | 110-41-8 | 3 | 0.030 |
| Acetaldehyde | | | | |
| Melonal | 1.000 | 106-72-9 | 3 | 0.670 |
| Flor Acetate | 11.800 | 5413-60-5 | 1 | 0.060 |
| Frutene | 7.000 | 17511-60-3 | 4 | 0.020 |
| Helional | 5.000 | 1205-17-0 | 2 | 0.0005 |
| Bourgeonal | 2.000 | 18127-01-0 | 4 | 0.004 |
| Linalool | 10.000 | 78-70-6 | 3 | 0.050 |
| Benzaldehyde | 0.200 | 100-52-7 | 1 | 1.110 |
| o-anisaldehyde | 15.000 | 135-02-4 | 1 | 0.320 |

Accords A, B, or C can be formulated in with other perfume raw materials in an amount, for example, of about 1%, by weight,of the malodor control composition. Additionally, the individual volatile aldehydes or a various combination of the volatile aldehydes can be formulated into the malodor control composition. In certain embodiments, the volatile aldehydes may be present in an amount up to 100%, by weight of the perfume mixture, alternatively from 1% to about 100%, alternatively from about 2% to about 100%, alternatively from about 3% to about 100%, alternatively from about 50% to about 100%, alternatively from about 70% to about 100%, alternatively from about 80% to about 100%, alternatively from about 1% to about 20%, alternatively from about 1% to about 10%, alternatively from about 1% to about 5%, alternatively from about 1% to about 3%, alternatively from about 2% to about 20%, alternatively from about 3% to about 20%, alternatively from about 4% to about 20%, alternatively from about 5% to about 20%.

In some embodiments where volatility is not important for neutralizing a malodor, the present invention may include poly-aldehydes, for example, di-, tri-, tetra-aldehydes. Such embodiments may include laundry detergents, additive, and the like for leave-on, through the wash, and rinse-off type of applications.

2. Low Molecular Weight Polyols

Low molecular weight polyols with relatively high boiling points, as compared to water, such as ethylene glycol, diethylene glycol, triethylene glycol, propylene glycol, dipropylene glycol, and/or glycerine may be utilized as a malodor counteractant for improving odor neutralization. Some polyols, e.g., dipropylene glycol, are also useful to facilitate the solubilization of some perfume ingredients in the composition of the present invention.

The glycol used in the composition of the present invention may be glycerine, ethylene glycol, propylene glycol, dipropylene glycol, polyethylene glycol, propylene glycol methyl ether, propylene glycol phenyl ether, propylene glycol methyl ether acetate, propylene glycol n-butyl ether, dipropylene glycol n-butyl ether, dipropylene glycol n-propyl ether, ethylene glycole phenyl ether, diethylene glycol n-butyl ether, dipropylene glycol n-butyl ether, diethylene glycol mono butyl ether, diprop_{y}lene glycol methyl ether, tripropylene glycol methyl ether, tripropylene glycol n-butyl ether, other glycol ethers, or mixtures thereof. In one embodiment, the glycol used is ethylene glycol, propylene glycol, or mixtures thereof. In another embodiment, the glycol used is diethylene glycol.

Typically, the low molecular weight polyol is added to the composition of the present invention at a level of from about 0.01% to about 5%, by weight of the composition, alternatively from about 0.05% to about 1%, alternatively from about 0.1% to about 0.5%, by weight of the composition. Compositions with higher concentrations may make fabrics susceptible to soiling and/or leave unacceptable visible stains on fabrics as the solution evaporates off of the fabric. When the malodor control polymer is a HMP, the weight ratio of low molecular weight polyol to the HMP is from about 500:1 to about 4:1, alternatively from about 1:100 to about 25:1, alternatively from about 1:50 to about 4:1, alternatively about 4:1.

3. Cyclodextrin

In some embodiments, the composition may include solubilized, water-soluble, uncomplexed cyclodextrin. As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in a donut-shaped ring. The specific coupling and conformation of the glucose units give the cyclodextrins a rigid, conical molecular structure with a hollow interior of a specific volume. The “lining” of the internal cavity is formed by hydrogen atoms and glycosidic bridging oxygen atoms, therefore this surface is fairly hydrophobic. The unique shape and physical-chemical property of the cavity enable the cyclodextrin molecules to absorb (form inclusion complexes with) organic molecules or parts of organic molecules which can fit into the cavity. Many perfume molecules can fit into the cavity.

Cyclodextrin molecules are described in U.S. Pat. No. 5,714,137, and U.S. Pat. No. 5,942,217. Suitable levels of cyclodextrin are from about 0.1% to about 5%, alternatively from about 0.2% to about 4%, alternatively from about 0.3% to about 3%, alternatively from about 0.4% to about 2%, by weight of the composition. Compositions with higher concentrations can make fabrics susceptible to soiling and/or leave unacceptable visible stains on fabrics as the solution evaporates off of the fabric. The latter is especially a problem on thin, colored, synthetic fabrics. In order to avoid or minimize the occurrence of fabric staining, the fabric may be treated at a level of less than about 5 mg of cyclodextrin per mg of fabric, alternatively less than about 2 mg of cyclodextrin per mg of fabric.

D. Acid Catalyst

The malodor control composition of the present invention may include an effective amount of an acid catalyst to neutralize sulfur-based malodors. It has been found that certain mild acids have an impact on aldehyde reactivity with thiols in the liquid and vapor phase. It has been found that the reaction between thiol and aldehyde is a catalytic reaction that follows the mechanism of hemiacetal and acetal formation path. When the present malodor control composition contains an acid catalyst and contacts a sulfur-based malodor, the volatile aldehyde reacts with thiol. This reaction may form a thiol acetal compound, thus, neutralizing the sulfur-based odor. Without an acid catalyst, only hemi-thiol acetal is formed.

Suitable acid catalysts have a VP, as reported by Scifinder, in the range of about 0.001 torr to about 38 torr, measured at 25° C., alternatively about 0.001 torr to about 14 torr, alternatively from about 0.001 to about 1, alternatively from about 0.001 to about 0.020, alternatively about 0.005 to about 0.020, alternatively about 0.010 to about 0.020.

The acid catalyst may be a weak acid. A weak acid is characterized by an acid dissociation constant, Kₐ, which is an equilibrium constant for the dissociation of a weak acid; the pKa being equal to minus the decimal logarithm of Kₐ. The acid catalyst may have a pKa from about 4.0 to about 6.0, alternatively from about 4.3 and 5.7, alternatively from about 4.5 to about 5, alternatively from about 4.7 to about 4.9. Suitable acid catalyst include those listed in Table 4.

TABLE 4

| | VP (torr) @ |
|---|---|
| Material | 25° C. |
| Formic Acid | 36.5 |
| Acetic Acid | 13.9 |
| Trimethyl Acetic Acid | 0.907 |
| Phenol (alkaline in liquid apps yet | 0.610 |
| acidic in vapor phase) | |
| Tiglic acid | 0.152 |
| Caprylic acid | 0.0222 |
| 5-Methyl thiophene carboxylic acid | 0.019 |
| Succinic acid | 0.0165 |
| Benzoic acid | 0.014 |
| Mesitylenic acid | 0.00211 |

Depending on the desired use of the malodor control composition, one may consider the scent character or the affect on the scent of the malodor control composition when selecting an acid catalyst. In some embodiments of the malodor control composition, it may be desirable to select an acid catalyst that provides a neutral to pleasant scent. Such acid catalysts may have a VP of about 0.001 torr to about 0.020 torr, measured at 25° C., alternatively about 0.005 torr to about 0.020 torr, alternatively about 0.010 torr to about 0.020 torr. Non-limiting examples of such acid catalyst include 5-methyl thiophene carboxaldehyde with carboxylic acid impurity, succinic acid, and benzoic acid.

The malodor control composition may include about 0.05% to about 5%, by weight of the malodor control composition, of an acid catalyst; alternatively about 0.1% to about 1.0%, alternatively about 0.1% to about 0.5%, alternatively about 0.1% to about 0.4%, alternatively about 0.4% to about 1.5%, alternatively about 0.4% of an acid catalyst.

In an acetic acid system, the present malodor control composition may include about 0.4% of acetic acid (50:50 TC:DPM, 0.4% acetic acid).

TABLE 5

| | Actual % | |
|---|---|---|
| | acetic | % Butanethiol |
| Sample Formulated | acid in DPM | reduction @ 30 min. |
| 50:50 TC:DPM 0% Acetic Acid | 0.00 | 12.00 |
| 50:50 TC:DPM 0.05% Acetic Acid | 0.04 | 14.65 |
| 50:50 TC:DPM 0.1% Acetic Acid | 0.10 | 25.66 |
| 50:50 TC:DPM 0.2% Acetic Acid | 0.42 | 34.68 |
| 50:50 TC:DPM 0.5% Acetic Acid | 1.00 | 24.79 |
| 50:50 TC:DPM 1.0% Acetic Acid | 2.00 | 7.26 |

When an acid catalyst is present with a volatile aldehyde (or RA), the acid catalyst may increase the efficacy of the volatile aldehyde on malodors in comparison to the malodor efficacy of the volatile aldehyde on its own. For example, 1% volatile aldehyde and 1.5% benzoic acid provides malodor removal benefit equal to or better than 5% volatile aldehyde alone.

The malodor control composition may have a pH from about 3 to about 8, alternatively from about 4 to about 7, alternatively from about 4 to about 6.

E. Buffering Agent

The composition of the present invention may include a buffering agent which may be a dibasic acid, carboxylic acid, or a dicarboxylic acid like maleic acid. The acid may be sterically stable, and used in this composition solely for maintaining the desired pH. The composition may have a pH from about 6 to about 8, alternatively from about 6 to about 7, alternatively about 7, alternatively about 6.5

Carboxylic acids such as citric acid may act as metal ion chelants and can form metallic salts with low water solubility. As such, in some embodiments, the freshening composition is essentially free of citric acids. The buffer can be alkaline, acidic or neutral.

Other suitable buffering agents for freshening compositions of this invention include biological buffering agents. Some examples are nitrogen-containing materials, sulfonic acid buffers like 3-(N-morpholino)propanesulfonic acid (MOPS) or N-(2-Acetamido)-2-aminoethanesulfonic acid (ACES), which have a near neutral 6.2 to 7.5 pKa and provide adequate buffering capacity at a neutral pH. Other examples are amino acids such as lysine or lower alcohol amines like mono-, di-, and tri-ethanolamine. Other nitrogen-containing buffering agents are tri(hydroxymethyl)amino methane (HOCH2)3CNH3 (TRIS), 2-amino-2-ethyl-1,3-propanediol, 2-amino-2-methyl-propanol, 2-amino-2-methyl-1,3-propanol, disodium glutamate, N-methyl diethanolamide, 2-dimethylamino-2-methylpropanol (DMAMP), 1,3-bis(methylamine)-cyclohexane, 1,3-diamino-propanol N,N-tetra-methyl-1,3-diamino-2-propanol, N,N-bis(2-hydroxyethyl)glycine (bicine) and N-tris (hydroxymethyl)methyl glycine (tricine). Mixtures of any of the above are also acceptable.

The composition of the present invention may contain at least about 0%, alternatively at least about 0.001%, alternatively at least about 0.01%, by weight of the composition, of a buffering agent. The composition may also contain no more than about 1%, alternatively no more than about 0.75%, alternatively no more than about 0.5%, by weight of the composition, of a buffering agent.

F. Solubilizer

The composition of the present invention may contain a solubilizing aid to solubilize any excess hydrophobic organic materials, particularly any perfume materials, and also optional ingredients (e.g., insect repelling agent, antioxidant, etc.) which can be added to the composition, that are not readily soluble in the composition, to form a clear solution. A suitable solubilizing aid is a surfactant, such as a no-foaming or low-foaming surfactant. Suitable surfactants are nonionic surfactants, cationic surfactants, amphoteric surfactants, zwitterionic surfactants, and mixtures thereof.

In some embodiments, the composition contains nonionic surfactants, cationic surfactants, and mixtures thereof. In one embodiment, the composition contains hydrogenated castor oil. One suitable hydrogenated castor oil that may be used in the present composition is Basophor™, available from BASF.

Compositions containing anionic surfactants and/or detergent surfactants may make fabrics susceptible to soiling and/or leave unacceptable visible stains on fabrics as the solution evaporates off of the fabric. In some embodiments, the composition is free of anionic surfactants and/or detergent surfactants.

When the solubilizing agent is present, it is typically present at a level of from about 0.01% to about 3%, alternatively from about 0.05% to about 1%, alternatively from about 0.01% to about 0.05%, by weight of the composition. Compositions with higher concentrations may make fabrics susceptible to soiling and/or leave unacceptable visible stains on fabrics as the solution evaporates off of the fabric.

G. Antimicrobial Compounds

The composition of the present invention may include an effective amount of a compound for reducing microbes in the air or on inanimate surfaces. Antimicrobial compounds are effective on gram negative and gram positive bacteria and fungi typically found on indoor surfaces that have contacted human skin or pets such as couches, pillows, pet bedding, and carpets. Such microbial species include Klebsiella pneumoniae, Staphylococcus aureus, Aspergillus niger, Klebsiella pneumoniae, Steptococcus pyogenes, Salmonella choleraesuis, Escherichia coil, Trichophyton mentagrophytes, and Pseudomonoas aeruginosa. In some embodiments, the antimicrobial compounds are also effective on viruses such H1-N1, Rhinovirus, Respiratory Syncytial, Poliovirus Type 1, Rotavirus, Influenza A, Herpes simplex types 1 & 2, Hepatitis A, and Human Coronavirus.

Antimicrobial compounds suitable in the composition of the present invention can be any organic material which will not cause damage to fabric appearance (e.g., discoloration, coloration such as yellowing, bleaching). Water-soluble antimicrobial compounds include organic sulfur compounds, halogenated compounds, cyclic organic nitrogen compounds, low molecular weight aldehydes, quaternary compounds, dehydroacetic acid, phenyl and phenoxy compounds, or mixtures thereof.

In one embodiment, a quaternary compound is used. Examples of commercially available quaternary compounds suitable for use in the composition is Barquat available from Lonza Corporation; and didecyl dimethyl ammonium chloride quat under the trade name Bardac® 2250 from Lonza Corporation.

The antimicrobial compound may be present in an amount from about 500 ppm to about 7000 ppm, alternatively about 1000 ppm to about 5000 ppm, alternatively about 1000 ppm to about 3000 ppm, alternatively about 1400 ppm to about 2500 ppm, by weight of the composition.

H. Preservatives

The composition of the present invention may include a preservative. The preservative is included in the present invention in an amount sufficient to prevent spoilage or prevent growth of inadvertently added microorganisms for a specific period of time, but not sufficient enough to contribute to the odor neutralizing performance of the composition. In other words, the preservative is not being used as the antimicrobial compound to kill microorganisms on the surface onto which the composition is deposited in order to eliminate odors produced by microorganisms. Instead, it is being used to prevent spoilage of the composition in order to increase shelf-life.

The preservative can be any organic preservative material which will not cause damage to fabric appearance, e.g., discoloration, coloration, bleaching. Suitable water-soluble preservatives include organic sulfur compounds, halogenated compounds, cyclic organic nitrogen compounds, low molecular weight aldehydes, parabens, propane diaol materials, isothiazolinones, quaternary compounds, benzoates, low molecular weight alcohols, dehydroacetic acid, phenyl and phenoxy compounds, or mixtures thereof.

Non-limiting examples of commercially available water-soluble preservatives for use in the present invention include a mixture of about 77% 5-chloro-2-methyl-4-isothiazolin-3-one and about 23% 2-methyl-4-isothiazolin-3-one, a broad spectrum preservative available as a 1.5% aqueous solution under the trade name Kathon® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from [nolex; 1,1′-hexamethylene bis(5-(p-chlorophenyl)biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name Glydant Plus® from Lonza; N-[1,3-bis(hydroxymethyl)2,5-dioxo-4-imidazolidinyl]-N,N′-bis(hydroxy-methyl) urea, commonly known as diazolidinyl urea, available under the trade name Germall® II from Sutton Laboratories, Inc.; N,N″-methylenebis{N′41-(hydroxymethyl)-2,5-dioxo-4-imidazolidinyl]urea}, commonly known as imidazolidinyl urea, available, e.g., under the trade name Abiol® from 3V-Sigma, Unicide U-13® from Induchem, Germall 115® from Sutton Laboratories, Inc.; polymethoxy bicyclic oxazolidine, available under the trade name Nuosept® C from Hills America; formaldehyde; glutaraldehyde; polyaminopropyl biguanide, available under the trade name Cosmocil CQ® from ICI Americas, Inc., or under the trade name Mikrokill® from Brooks, Inc; dehydroacetic acid; and benzsiothiazolinone available under the trade name Koralone™ B-119 from Rohm and Hass Corporation.

Suitable levels of preservative are from about 0.0001% to about 0.5%, alternatively from about 0.0002% to about 0.2%, alternatively from about 0.0003% to about 0.1%, by weight of the composition.

I. Wetting Agent

The composition may include a wetting agent that provides a low surface tension that permits the composition to spread readily and more uniformly on hydrophobic surfaces like polyester and nylon. It has been found that the aqueous solution, without such a wetting agent will not spread satisfactorily. The spreading of the composition also allows it to dry faster, so that the treated material is ready to use sooner. Furthermore, a composition containing a wetting agent may penetrate hydrophobic, oily soil better for improved malodor neutralization. A composition containing a wetting agent may also provide improved “in-wear” electrostatic control. For concentrated compositions, the wetting agent facilitates the dispersion of many actives such as antimicrobial actives and perfumes in the concentrated aqueous compositions.

Non-limiting examples of wetting agents include block copolymers of EO and PO. Suitable block polyoxyethylene-polyoxypropylene polymeric surfactants include those based on ethylene glycol, propylene glycol, glycerol, trimethylolpropane and ethylenediamine as the initial reactive hydrogen compound. Polymeric compounds made from a sequential ethoxylation and propoxylation of initial compounds with a single reactive hydrogen atom, such as C₁₂₋₁₈ aliphatic alcohols, are not generally compatible with the cyclodextrin. Certain of the block polymer surfactant compounds designated Pluronic® and Tetronic® by the BASF-Wyandotte Corp., Wyandotte, Mich., are readily available.

Nonlimiting examples of cyclodextrin-compatible wetting agents of this type are described in U.S. Pat. No. 5,714,137 and include the Silwet® surfactants available from Momentive Performance Chemical, Albany, New York. Exemplary Silwet surfactants are as follows:

| Name | Average MW |
|---|---|
| L-7608 | 600 |
| L-7607 | 1,000 |
| L-77 | 600 |
| L-7605 | 6,000 |
| L-7604 | 4,000 |
| L-7600 | 4,000 |
| L-7657 | 5,000 |
| L-7602 | 3,000; |

and mixtures thereof.

J. Aqueous Carrier

The composition of the present invention may include an aqueous carrier. The aqueous carrier which is used may be distilled, deionized, or tap water. Water may be present in any amount for the composition to be an aqueous solution. In some embodiments, water may be present in an amount of about 50% to about 99.5%, alternatively about 85% to about 99.5%, alternatively about 90% to about 99.5%, alternatively about 92% to about 99.5%, alternatively about 95%, by weight of said composition. Water containing a small amount of low molecular weight monohydric alcohols, e.g., ethanol, methanol, and isopropanol, or polyols, such as ethylene glycol and propylene glycol, can also be useful. However, the volatile low molecular weight monohydric alcohols such as ethanol and/or isopropanol should be limited since these volatile organic compounds will contribute both to flammability problems and environmental pollution problems. If small amounts of low molecular weight monohydric alcohols are present in the composition of the present invention due to the addition of these alcohols to such things as perfumes and as stabilizers for some preservatives, the level of monohydric alcohol may be less than about 15%, alternatively less than about 6%, alternatively less than about 3%, alternatively less than about 1%, by weight of the composition.

K. Other Optional Ingredients

Adjuvants can be optionally added to the composition herein for their known purposes. Such adjuvants include, but are not limited to, water soluble metallic salts, antistatic agents, insect and moth repelling agents, colorants, antioxidants, and mixtures thereof.

### II. Method of Making

The composition can be made in any suitable manner known in the art. All of the ingredients can simply be mixed together. In certain embodiments, it may be desirable to make a concentrated mixture of ingredients and dilute by adding the same to an aqueous carrier before dispersing the composition into the air or on an inanimate surface. In another embodiment, the malodor control polymer may be dispersed in one vessel containing deionized water and ethanol, and low molecular weight polyols. To this vessel, then, the bufferis added until fully dispersed and visually dissolved. In a separate vessel, the solubilizer and perfume are mixed until homogenous. The solution of solubilizer and perfume are then added to the first mixing vessel, and mixed until homogenous.

### III. Methods of Use

The malodor control composition of the present invention may be used in a wide variety of applications that neutralize malodors in the vapor and/or liquid phase. In some embodiments, the malodor control composition may be formulated for use in energized vapor phase systems. “Energized” as used herein refers to a system that operates by using an electrical energy source, such as a battery or electrical wall outlet, to emit a targeted active. For such systems, the VP of the volatile aldehyes may be about 0.001 torr to about 20 torr, alternatively about 0.01 torr to about 10 torr, measured at 25° C. One example of an energized vapor phase system is a liquid electric plug-in type air freshening device.

In some embodiments, the malodor control composition may be formulated for use in non-energized vapor phase systems. “Non-energized” as used herein refers to a system that emits a targeted active passively or without the need for an electrical energy source. Aerosol sprayers and traditional trigger/pump sprayers are considered non-energized systems. For such non-energized systems, the VP of the volatile aldehydes may be about 0.01 torr to about 20 torr, alternatively about 0.05 torr to about 10 torr, measured at 25° C. Non-limiting examples of a non-energized vapor phase system are passive air freshening diffusers such as those known by the trade name Renuzit® Crystal Elements; and aerosol sprays such as fabric and air freshening sprays and body deodorants.

In other embodiments, the malodor control composition may be formulated for use in a liquid phase system. For such systems, the VP may be about 0 torr to about 20 torr, alternatively about 0.0001 torr to about 10 torr, measured at 25° C. Non-limiting examples of a liquid phase system are liquid laundry products, such as laundry detergents and additives; dish detergents; personal hygiene products such as body washes, shampoos, conditioners.

The malodor control composition may also be formulated for use in substrates such as plastics, wovens, or non-wovens (e.g cellulose fibers for paper products). Such substrates may be used as pet food packaging; paper towels; tissues; trash bags; diapers; baby wipes; adult incontinence products; feminine hygiene products such as sanitary napkins and tampons. The malodor control composition may also be formulated for use in commercial or industrial systems such as in septic tanks or sewage treatment equipment.

The malodor control composition of the present invention can be used by dispersing, e.g., by placing an aqueous solution into a dispensing means, such as a spray dispenser and spraying an effective amount into the air or onto the desired surface or article. An effective amount as defined herein means an amount sufficient to neutralize malodor to the point that it is not discernible by the human sense of smell yet not so much as to saturate or create a pool of liquid on an article or surface and so that, when dry, there is no visual deposit readily discernible. Dispersing can be achieved by using a spray device, a roller, a pad, etc.

The present invention encompasses the method of dispersing an effective amount of the composition onto household surfaces. The household surfaces are selected from the group consisting of countertops, cabinets, walls, floors, toilets, bathroom surfaces, and kitchen surfaces.

The present invention encompasses the method of dispersing a mist of an effective amount of the composition onto fabric and/or fabric articles. The fabric and/or fabric articles include, but are not limited to, clothes, curtains, drapes, upholstered furniture, carpeting, bed linens, bath linens, tablecloths, sleeping bags, tents, car interior, e.g., car carpet, fabric car seats, etc.

The present invention encompasses the method of dispersing a mist of an effective amount of the composition onto and into shoes wherein the shoes are not sprayed to saturation.

The present invention encompasses the method of dispersing a mist of an effective amount of the composition onto shower curtains.

The present invention relates to the method of dispersing a mist of an effective amount of the composition onto and/or into garbage cans and/or recycling bins.

The present invention relates to the method of dispersing a mist of an effective amount of the composition into the air to neutralize malodor.

The present invention relates to the method of dispersing a mist of an effective amount of the composition into and/or onto major household appliances including, but not limited to, refrigerators, freezers, washing machines, automatic dryers, ovens, microwave ovens, dishwashers, etc., to neutralize malodor.

The present invention relates to the method of dispersing a mist of an effective amount of the composition onto cat litter, pet bedding and pet houses to neutralize malodor.

The present invention relates to the method of dispersing a mist of an effective amount of the composition onto household pets to neutralize malodor.

### EXAMPLES

### Aqueous Composition

Table 6 shows non-limiting examples of compositions according to the present invention. A mixture of water, ethanol, and Silwet L-7600 surfactant is prepared by mixing. The final pH is adjusted to 7 using 30% maleic acid and this solution is used as Control 1. Control 2 and Test Solutions I-IV are prepared by adding desired ingredients right before adjusting the pH.

TABLE 6

| | | | | | Test Solution | Test |
|---|---|---|---|---|---|---|
| | | | Test | Test | III | Solution IV |
| | Control 1 | Control 2 | Solution I | Solution II | (Zn-HMP | (Zn-HMP |
| Ingredient | (Blank) | (CD) | (HMP) | (HMP) | complex) | complex) |
| Ethanol | 3 | 3 | 3 | 3 | 3 | 3 |
| Surfactant | 0.1 | 0.1 | 0.1 | 0.1 | 0.1 | 0.1 |
| (Silwet L-7600) | | | | | | |
| Hydroxypropyl | — | 0.5 | — | — | — | — |
| Beta CD | | | | | | |
| HMP | — | — | 0.5 | 0.5 | — | — |
| Zn-HMP | — | — | — | — | 0.7 | 0.7 |
| Maleic Acid | As needed | As needed | As needed | As needed | As needed | As needed |
| Perfume | — | — | 0.05 | 1.0 | 0.05 | 1.0 |
| comprising a | | | | | | |
| volatile aldehyde | | | | | | |
| mixture | | | | | | |
| Water | Balance | Balance | Balance | Balance | Balance | Balance |
| Total | 100 | 100 | 100 | 100 | 100 | 100 |
| Final pH | 7 | 7 | 7 | 7 | 7 | 7 |

### Formulation of Metal Polymer Coordination Complexes

This example illustrates the preparation of the present invention containing water soluble zinc-polymer coordination complexes.

A 50 ml mixture of water, ethanol, and Silwet L-7600 surfactant was prepared by mixing. Separately, 50 ml aqueous solution of zinc polymer coordination complexes were prepared by stirring 0.2% ZnCl2 and 0.5% polymer for 30 minutes in water. Finally, the solutions were combined and the solution pH was adjusted to 7 using 30% maleic acid. Two blank solutions (pH 5 and pH 7) were used as representative Controls. Control 3 contained ZnCl2 at pH 5 since at a higher pH, ZnCl2 solutions are not stable.

TABLE 7

| | | | I |
|---|---|---|---|
| | Control 1 | Control 3 | (Zn-polymer |
| Ingredient | (Blank) | (ZnCl₂) | complexes) |
| Water | 96.85 | 95.9 | 96.2 |
| Ethanol | 3 | 3 | 3 |
| Surfactant | 0.1 | 0.1 | 0.1 |
| (Silwet L-7600) | | | |
| ZnCl2 | — | 1.0 | 0.2 |
| Polymer | — | — | 0.5 |
| Maleic Acid | as needed | as needed | as needed |
| Sodium | as needed | as needed | — |
| hydroxide | | | |
| Total | 100 | 100 | 100 |
| Final pH | 7 | 5 | 7 |

### Malodor Control Performance of HMPs

This example illustrates the malodor efficacy of the HMPs of the present invention. Isovaleric acid was chosen as a chemical surrogate for body odor while butylamine was used as a representative for amine-containing odors such as fish, pet urine, etc. Hydrophobic greasy cooking odors were represented by aldehydes such as nonanal.

5 ml test solution was placed in a GC-MS vial and spiked with 5 microliters of chemical surrogates shown in Table 8. The solutions are first equilibrated at room temperature for 2 hours, then incubated at 35° C. for 30 minutes. The headspace of each vial is finally sampled using a polydimethyl siloxane (PDMS) / Solid-Phase-Micro-Extraction (SPME) fiber and analyzed by GC/MS. The reductions in head space concentrations of odor molecules are measured and the data are normalized to Control.

Results are shown in Table 3. Lower numbers denote high levels of malodor molecules present in the solution that are attributed to high malodor control efficacy of polymers. Table 8 demonstrates that HMPs and metallated polymers have broader malodor removal efficacy over the Controls and unmodified polymers.

TABLE 8

| | Odor Molecules | | |
|---|---|---|---|
| | Isovaleric Acid | Butylamine | Nonanal |
| Technology | (Body) | (Fish) | (Grease) |
| Control 1 | 1.0 | 1.0 | 1.0 |
| Control 2 | 0.67 | 1.0 | 0.48 |
| Hydroxypropyl Beta CD | | | |
| Lupasol WF | 0.1 | 0.01 | 0.78 |
| PEI 25,000 (no hydrophobic | | | |
| modification) | | | |
| 100% | 0.77 | 1.0 | 0.87 |
| ethyleneoxide/ | | | |
| propyleneoxidemodified | | | |
| PEI 600 | | | |
| Lupamin 9000 (PVA) | 0.93 | 0.97 | 0.96 |
| (0% hydrolyzed) | | | |
| Lupamin 9030 | 0.61 | 0.06 | 0.05 |
| (30% hydrolyzed) | | | |
| Lupamin 9095 | 0.37 | 0.01 | 0.04 |
| (95% hydrolyzed) | | | |
| Lupamin 1595 | 0.26 | 0.01 | 0.02 |
| (95% hydrolyzed) | | | |
| 25% C6 modified PEI 1800 | 0.02 | 0.01 | 0.37 |
| ZnCl2 (pH 5) | 1.0 | 0.0 | 1.0 |
| Zn-Lupasol WF complex | 0.02 | 0.11 | 0.87 |
| (polymer/ZnCl2 = 2.5) | | | |
| Zn-Lupamin 1595 complex | 0.01 | 0.00 | 0.03 |
| (polymer/ZnCl2 = 2.5) | | | |

### Sulfur Odor Control Performance of Zinc Polymer Complexes

This example illustrates the sulfur odor efficacy of water soluble zinc polymer coordination complexes of the present invention.

Butanethiol and dipropoyl sulfide were chosen as chemical surrogates for sulfur containing odors such as kitchen (onion/garlic), sewage, etc. These two molecules also enable the assessment of efficacy of polymer for sulfur molecules having different degrees of hydrophobicity (e.g more hydrophobic dipropylsulfide is usually harder to mitigate with hydrophilic technologies such as cyclodextrin).

5 ml test solution was placed in a GC-MS vial and spiked with 3 parts-per-million of butanethiol or dipropylsulfide. The solutions were first equilibrated at room temperature for 2 hours, then incubated at 35° C. for 30 minutes. The headspace of each vial was finally sampled using a PDMS/SPME fiber and analyzed by GC/MS. The reductions in head space concentrations of sulfur molecules were measured and the data were normalized to Control (Table 9).

TABLE 9

| Technology | Butanethiol | Dipropylsulfide |
|---|---|---|
| Control | 1.0 | 1.0 |
| Lupasol WF | 1.0 | 1.0 |
| Lupamin 1595 | 1.0 | |
| (95% hydrolyzed) | | |
| ZnCl2 (pH 5) | 0.8 | 1.0 |
| Zn-Lupasol WF complex | 0.5 | 1.0 |
| (Zn/polymer = 0.2) | | |
| Zn-Lupamin 1595 complex | 0.4 | — |
| (Zn/polymer = 0.2) | | |
| Zn—C 12 modified PEI25000 complex | 0.01 | <0.7 |
| (Zn/polymer = 0.2) | | |

### Odor Prevention Performance of Zinc Polymer Complexes

This Example illustrates the odor prevention efficacy of water soluble zinc polymer coordination complexes of the present invention.

The Control formulation containing no malodor control polymer or zinc salt, and Formulations containing individual polymers, zinc salts, and zinc-polymer complexes are compared for their effect on odor prevention, through microbe reduction.

Soiled sponge samples were cut into 1×6 cm strips and treated with the solutions (Table 10) for 15 minutes and dried at ambient temperature for 12 hours. The treated 1 cm strips were then cut into lx1 cm pieces, placed into SOLARIS scintillation vials, and 1 ml of MOPS buffer was added. The open vials were placed into outer SOLARIS vials containing thymolphthalein blue pH indicator and the vials were finally capped. The sealed vials were placed into a SOLARIS machine and incubated for 120 hrs at 37° C. Colorimetric measurements were conducted according to SOLARIS VIV protocol and the detection times of acidic respiratory byproducts were record (Table 10).

TABLE 10

| | Respiratory Byproducts |
|---|---|
| Technology | Detection Time (hrs) |
| Control | 2.35 |
| ZnCl2 | 3.38 |
| Lupamin 1595 | 15.5 |
| Zn-Lupamin 1595 complex | not detected |
| (ZnCl2/polymer = 0.4) | |

### Analytical Test—Effect of Volatile Aldehydes on Amine-Based and Sulfur-Based Malodors

Malodor standards are prepared by pipeting 1 mL of butylamine (amine-based malodor) and butanethiol (sulfur-based malodor) into a 1.2 liter gas sampling bag. The bag is then filled to volume with nitrogen and allowed to sit for at least 12 hours to equilibrate.

A 1 μL sample of each volatile aldehyde listed in Table 11 and of each Accord (A, B, and C) listed in Tables 1 to 3 is pippeted into individual 10 mL silanized headspace vials. The vials are sealed and allowed to equilibrate for at least 12 hours. Repeat 4 times for each sample (2 for butylamine analysis and 2 for butanethiol analysis).

After the equilibration period, 1.5 mL of the target malodor standard is injected into each 10 mL vial. For thiol analysis, the vials containing a sample +malodor standard are held at room temperature for 30 minutes. Then, a 1 mL headspace syringe is then used to inject 250 μL of each sample/malodor into a GC/MS split/splitless inlet. For amine analysis, a 1 mL headspace syringe is used to inject 500 μL of each sample/malodor immediately into the GC/MS split/splitless inlet. A GC pillow is used for the amine analysis to shorten the run times.

Samples are then analyzed using a GC/MS with a DB-5, 20 m, 1 μm film thickness column with an MPS-2 autosampler equipment with static headspace function. Data is analyzed by ion extraction on each total ion current (56 for thiol and 30 for amine) and the area is used to calculate the percent reduction from the malodor standard for each sample.

Table 11 shows the effect of certain volatile aldehydes on neutralizing amine-based and sulfur based malodors at 40 seconds and 30 minutes, respectively.

TABLE 11

| | At least 20% | |
|---|---|---|
| | butylamine | At least 20% |
| | reduction at | butanethiol |
| Perfume Raw Material (R-CHO) | 40 secs.? | reduction at 30 mins.? |
| 2,4,5 Trimethoxy Benzaldehyde | No | No |
| 2,4,6-Trimethoxy-benzylaldehyde | No | No |
| 2-ethoxy benzylaldehyde | Yes | Yes |
| 2-isopropyl-5-methyl-2-hexenal | Yes | Yes |
| 2-methyl-3-(2-furyl)-propenal | No | No |
| 3,4,5 Trimethoxy Benzaldehyde | No | No |
| 3,4-Trimethoxy-benzylaldehyde | No | No |
| 4-tertbutyl benzylaldehyde | Yes | No |
| 5-methyl furfural | Yes | Yes |
| 5-methyl-thiophene- | No | Yes |
| carboxaldehyde | | |
| Adoxal | Yes | No |
| Amyl cinnamic aldehyde | No | No |
| Benzylaldehyde | Yes | No |
| Bourgenal | No | Yes |
| Cinnamic aldehyde | Yes | Yes |
| Citronelyl Oxyacetaldehyde | No | No |
| Cymal | Yes | No |
| Decyl aldehyde | Yes | No |
| Floral Super | Yes | Yes |
| Florhydral | Yes | Yes |
| Floralozone | No | No |
| Helional | Yes | No |
| Hydroxycitronellal | No | No |
| Lauric aldehyde | Yes | No |
| Ligustral | Yes | No |
| Lyral | Yes | No |
| Melonal | Yes | No |
| Methyl nonyl acetaldehyde | No | No |
| o-anisaldehyde | Yes | Yes |
| p-anisaldehyde | Yes | No |
| Pino acetaldehyde | Yes | Yes |
| P.T. Bucinal | Yes | No |
| Thiophene Carboxaldehyde | Yes | No |
| Trans-4-decenal | Yes | Yes |
| Trans Trans 2,4-Nonadienal | Yes | No |
| Undecyl aldehyde | Yes | No |

Table 12 shows the percent reduction of butylamine and butaniethiol at 40 seconds and 30 minutes, respectively, for Accords A, B, and C.

TABLE 12

| | % reduction of butylamine at | % reduction of butanethiol at |
|---|---|---|
| Accord | 40 secs. | 30 mins. |
| Accord A | 76.58 | 25.22 |
| Accord B | 51.54 | 35.38 |
| Accord C | 65.34 | 24.98 |

### Analytical Test—Effect of Acid Catalysts on Sulfur-Based Malodors

The above analytical test is repeated using samples containing an acid catalyst to test their effect on sulfur-based malodors. Specifically, a 1μL aliquot of each of the following controls and acid catalyst samples are pipeted into individual 10 mL silanized headspace vials in duplicate: thiophene carboxyaldehyde as a control; a 50/50 mixture of thiophene carboxaldehyde and each of the following acid catalysts at 0.04%, 0.10%, 0.43% in DPM, 1.02% in DPM, and 2.04% in DPM: phenol, mesitylenic acid, caprylic acid, succinic acid, pivalic acid, tiglic acid, and benzoic acid.

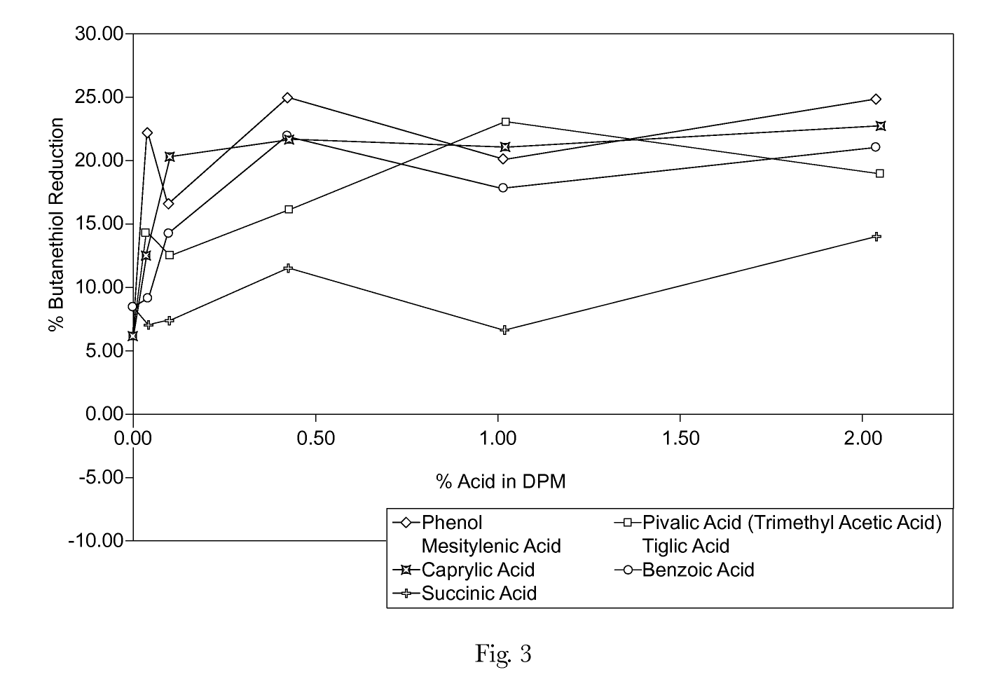

FIG. 3 demonstrates that low vapor pressure acid catalysts provide up to 3 times better reduction of sulfur-based malodors in comparison to the control.

### Analytical Test—Effect of Volatile Aldehydes and Acid Catalyst on Amine-Based and Sulfur-Based Malodors

The above analytical test is repeated using sample formulations containing volatile aldehydes (or RA) and an acid catalyst, as outlined in Tables 13 and 14.

Tables 13 and 14 show that a perfume mixture having as little as 1% volatile aldehyde along with 1.5% acid catalyst performs better at reducing butylamine and butanethiol than the same perfume mixture having 5% volatile aldehyde.

TABLE 13

| | % butylamine | | % butanethiol | |
|---|---|---|---|---|
| | reduction at | | reduction | |
| Formulation | 40 secs. | | at 30 mins. | |
| Perfume Mixture w/ 5% RA | 34.21 | — | 2.40 | — |
| (Control) | | | | |
| Perfume Mixture w/ 1% RA and | 41.63 | +7.42 | 11.95 | +9.55 |
| w/ 1.5% Benzoic Acid | | | | |
| Perfume Mixture w/ 3% RA and | 36.19 | +1.98 | 13.56 | +11.16 |
| w/ 1.5% Benzoic Acid | | | | |
| Perfume A Mixture w/ 5% RA and | 41.26 | +7.05 | 9.56 | +5.02 |
| w/ 1.5% Benzoic Acid | | | | |

TABLE 14

| | % butylamine | | % butanethiol | |
|---|---|---|---|---|
| | Reduction at | | reduction at | |
| Formulation | 40 secs. | | 30 mins. | |
| Perfume mixture w/ 5% RA | 4.94 | — | 10.52 | — |
| (Control) | | | | |
| Perfume mixture w/ 1% RA and | 11.61 | +6.67 | 18.82 | +8.30 |
| w/ 1.5% Benzoic Acid | | | | |
| Perfume mixture w/ 3% RA and | 26.89 | +21.95 | 14.85 | +4.33 |
| w/ 1.5% Benzoic Acid | | | | |
| Perfume mixture w/ 5% RA and | 20.27 | +15.33 | 16.84 | +6.32 |
| w/ 1.5% Benzoic Acid | | | | |

### Sensory Test—Effect of Volatile Aldehydes on a Sulfur-Based Malador

Place Presto™ skillet into fume hood and turn on to 250° F. Place 80 grams of Crisco® oil into skillet and cover with skillet lid. Allow 10 minutes for equilibration. Remove skillet lid and check oil temperature with thermometer. Place 50 grams of chopped, commercially prepared garlic in water into skillet. Cover skillet with lid. Cook for 2.5 minutes or until garlic is translucent, with a portion staring to turn brown but not burn. Remove garlic from the skillet. Place 5 grams of garlic in each of 4 Petri dishes. Place covers on each Petri dish.

Place each covered Petri dish into individual test chambers. Each test chamber is 39.25 inches wide, by 25 inches deep, by 21.5 inches high with a volume of 12.2 cubic feet (0.34 cubic meters). The test chamber can be purchased from Electro-Tech Systems, Glenside, PA. Each test chamber is equipped with a fan (Newark catalog #70K9932, 115 VAC, 90CFM) purchased from Newark Electronics, Chicago, Ill.

Remove the lids of the Petri dishes to expose the malodor for a dwell time sufficient to provide an initial odor intensity grade of 70-80 (about 1 minute). Once the initial odor intensity grade has been reached in a test chamber, remove the Petri dish from the test chamber.

Next, 3 Febreze® Noticeables™ air freshening devices, marketed by The Procter and

Gamble Company, are each filled with the Control composition shown in Table 15.

TABLE 15
Control Composition

| Material Name | Wt % |
|---|---|
| Benzaldehyde | 0.150 |
| Floralozone | 0.097 |
| Helional | 1.455 |
| Hydroxycitronellal | 3.880 |
| Ligustral Or Triplal | 1.028 |
| Esters | 12.950 |
| Ethers | 50.190 |
| Ketones | 3.010 |
| Lactones | 0.490 |
| Alcohols | 21.610 |
| Terpenes | 5.140 |

The devices are set to the low intensity position and plugged into 3 of the 4 test chambers. All doors on chamber are closed.

At 5, 15, 20, 30, 45, and 60 minutes, trained evaluators open each chamber, smell the chamber for malodor intensity, and assign a malodor score, based on the scale in Table 16. The chamber door is closed but not locked between sequential evaluators. The scores are tabulated and the average score for each time interval is recorded.

TABLE 16
Expert Sensory Grader Malodor Evaluation Scale

| Score | Description corresponding to Score |
|---|---|
| 0 | No malodor present |
| 10 | Very slight malodor - “I think there is a malodor |
| | present.” |
| 20 | Slight malodor - “I detect something but cannot |
| | identify specific malodor.” |
| 25 | Slight malodor |
| 50 | Moderate |
| 75 | Strong Malodor |
| 100 | Extremely Strong Malodor |

The above protocol is repeated using Prototype 1 shown in Table 17 (instead of Control composition in Table 15).

TABLE 17
Prototype 1

| Material Name | Wt. % |
|---|---|
| Benzaldehyde | 0.135 |
| Floralozone | 0.087 |
| Helional | 1.310 |
| Hydroxycitronellal | 3.492 |
| Ligustral Or Triplal | 0.925 |
| o-anisaldehyde | 2.500 |
| Intreleven Aldehyde | 0.500 |
| Florhydral | 1.000 |
| Floral Super | 2.500 |
| Scentenal | 1.000 |
| Cymal | 2.500 |
| Esters | 11.662 |
| Ethers | 45.171 |
| Ketones | 2.705 |
| Lactones | 0.437 |
| Alcohols | 19.446 |
| Terpenes | 4.632 |

The above protocol is repeated using Prototype 2 shown in Table 18.

TABLE 18
Protoype 2

| Material Name | Wt. % |
|---|---|
| Benzaldehyde | 0.135 |
| Floralozone | 0.087 |
| Helional | 1.310 |
| Hydroxycitronellal | 3.492 |
| Ligustral Or Triplal | 0.925 |
| o-anisaldehyde | 2.250 |
| Intreleven Aldehyde | 0.450 |
| Florhydral | 0.900 |
| Floral Super | 2.250 |
| Scentenal | 0.900 |
| Cymal | 2.250 |
| 5-Methyl Thiophene Carboxaldehyde | 1.000 |
| Esters | 11.662 |
| Ethers | 45.171 |
| Ketones | 2.705 |
| Lactones | 0.437 |
| Alcohols | 19.446 |
| Terpenes | 4.632 |

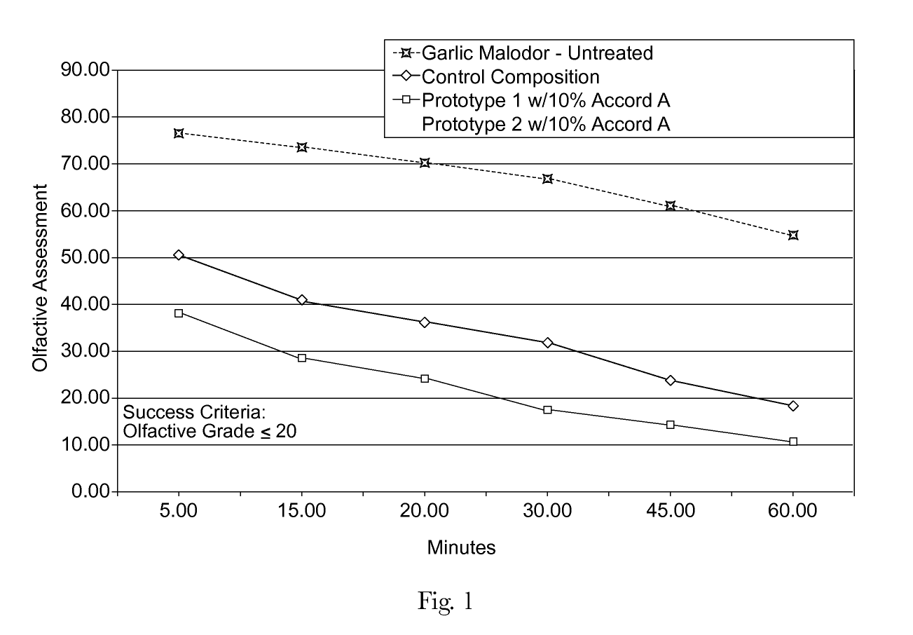

FIG. 1 shows that a protoype perfume formulations having 10% of the volatile aldehydes of the present invention reduces the garlic malodor more than the Control composition that lacks such malodor control composition.

### Sensory Test—Effect of Volatile Aldehydes on an Amine-Based Malodor

Separate fresh ocean perch fillets from skin and add to a Magic Bullet™ food chopper. Fish meat is chopped for 35-40 seconds. 25 grams of chopped fish is weighed and fashioned into a patty suitable to fit into a 60×15 mm Petri dish. Repeat 3 more times so there is one fish patty in each of 4 Petri dishes. Add 40 g of Crisco® oil to Presto™ skillet. Place lid on skillet and turn on to 350° F. Allow 10 minutes for equilibration. Remove lid. Cut a slit in the middle of each patty, place 1 patty into skillet, and begin frying. Replace lid. After 2.5 minutes, flip fish patty and fry an additional 2.5 minutes. Remove fish patty from skillet and blot briefly onto a paper towel for 10 seconds. Fry the remaining 3 patties in the same manner. Place each fish patty into a 60×15 mm Petri dish and cover with a lid.

Introduce each Petri dish containing a fish patty into individual test chambers. The specifications of the test chamber are the same as those in the above sulfur-based (i.e. garlic) malodor test. Remove the lids to expose the malodor for a dwell time sufficient for providing an initial odor intensity grade of 70-80 (about 1 minute). Once the initial odor intensity grade has been reached in a test chamber, remove the Petri dish from the test chamber.

Next, 3 Febreze Noticeables air freshening devices, marketed by The Procter and Gamble Company, are each filled with the Control composition outlined in Table 15. The devices are set to the low intensity position and plugged into 3 of the 4 test chambers. All doors on chamber are closed.

At 5, 15, 20, 30, 45, and 60 minutes, trained evaluators open each chamber, smell the chamber for malodor intensity, and assign a malodor score, based on the scale in Table 14. The chamber door is closed but not locked between sequential evaluators. The scores are tabulated and the average score for each time interval is recorded.

The above protocol is repeated using Prototype 1 shown in Table 17 (instead of Control composition); and then using Prototype 2 shown in Table 18.

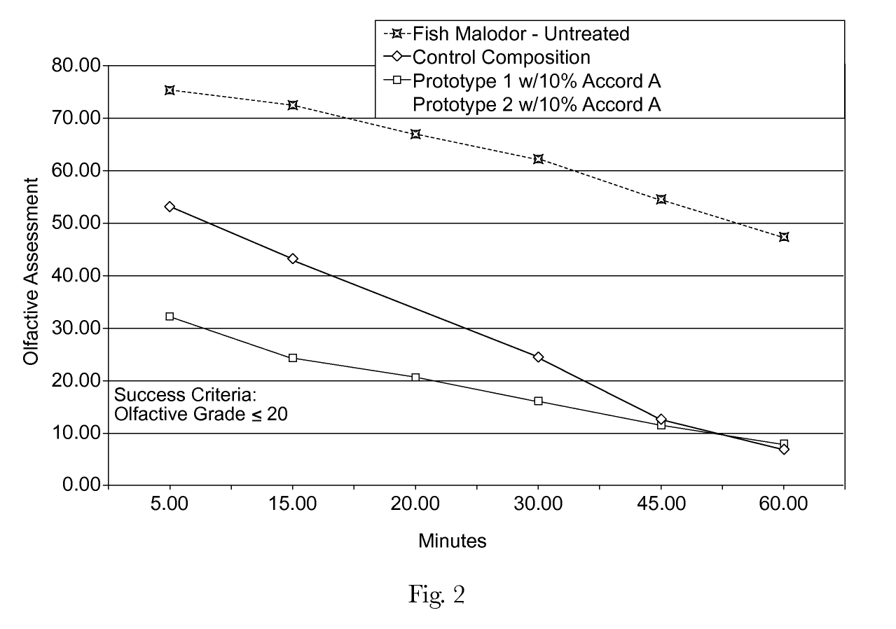

FIG. 2 shows that a prototype perfume formulation having 10% of the malodor control composition of the present invention reduces the fish malodor more than the Control that lacks such malodor control composition.

### Sensory Test—Effect of Volatile Aldehyde and Acid Catalyst on an Amine-Based and Sulfur-Based Malodors

The above sensory test protocols for amine-based and sulfur-based malodors are repeated using the 2 formulations outlined in Table 19; and then using the 2 formulations outlined in Table 20, except the Table 20 formulations are separately loaded into 2 Febreze® Set & Refresh passive air fresheners, marketed by The Procter and Gamble Company (vs. Febreze Noticeables devices). Results are shown in Tables 19 and 20.

Tables 19 and 20 demonstrates that perfume formulations with 1% volatile aldehyde and 1.5% acid catalyst provide malodor removal benefit equal to or better than perfume formulations with 5% volatile aldehyde alone.

TABLE 19
Sensory testing for malodor reduction with Febreze Noticeables
(10 replicates per test):

| | Amine - Fish | | Thiol - Garlic | |
|---|---|---|---|---|
| Formulation | Malodor Removal | | Malodor Removal | |
| Perfume mixture w/ 5% RA | *20 mins. | — | *22 mins. | — |
| (Control) | | | | |
| Perfume mixture w/ 1% RA and | *16 mins. | +4 | *20 mins. | +2 |
| w/ 1.5% benzoic acid | | | | |

*Meets Success Criteria: Olfactive Grade <20 at defined time.

TABLE 20
Sensory testing for malodor reduction with Febreze Set & Refresh
(10 replicates per test):

| | |
|---|---|
| | Amine - Fish |
| Formulation | Malodor Removal |

| | | |
|---|---|---|
| Perfume mixture w/ 5% RA | *23 mins. | — |
| (Control) | | |
| Perfume mixture w/ 1% RA and | *12 mins. | +11 |
| w/ 1.5% benzoic acid | | |

*Meets Success Criteria: Olfactive Grade <20 at defined time.

Throughout this specification, components referred to in the singular are to be understood as referring to both a single or plural of such component.

All percentages stated herein are by weight unless otherwise specified.

Every numerical range given throughout this specification will include every narrower numerical range that falls within such broader numerical range, as if such narrower numerical range were all expressly written herein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 6.1, 3.5 to 7.8, 5.5 to 10, etc.

Further, the dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A composition for reducing malodor comprising:
(a) an effective amount of malodor control polymer selected from the group consisting of:
(i) a metallated malodor control polymer comprising a water-soluble metal ion and a polymer selected from the group consisting of: partially hydrolyzed PVam, partially hydrolyzed hydrophobically modified PVam, PEI, hydrophobically modified PEI, PAMam, hydrophobically modified PAMam, PAam, hydrophobically modified PAam, PEam, hydrophobically modified PEam, and mixtures thereof; and
(ii) a hydrophobically modified polymer having structure (I):
P(R)x (I)
wherein:
P is selected from the group consisting of: partially hydrolyzed PVams, PEIs, PAMams, PAams, PEams, and mixtures thereof;
x is degree of substitution of the amine sites on the polymer and is less than 100%; and
R is a C2 to C26 alkyl or alkenyl; and
(iii) mixtures thereof;
(b) a malodor counteractant comprising a perfume mixture comprising at least on volatile aldehyde;
(c) an acid catalyst; and
(d) an aqueous carrier;
wherein said composition comprises a pH of about 5 to about 10.

2. The composition of claim 1 wherein said malodor control polymer is a hydrophobically modified polymer.

3. The composition of claim 2 wherein said hydrophobically modified polymer is hydrophobically modified, 95% hydrolyzed PVam.

4. The composition claim 2 wherein said hydrophobically modified polymer is a hydrophobic ally modified PEI.

5. The composition of claim 2 wherein R is a C4-C10 alkyl or alkylene.

6. The composition of claim 2 wherein R is C16 to C26 alkyl or alkylene.

7. The composition of claim 2 wherein R is a C6 alkyl or alkylene.

8. The composition of claim 4 wherein said hydrophobically modified PEI is a homopolymeric polyethyleneimine having a molecular weight of about 10,000 to about 350,000 Daltons.

9. The composition of claim 1 wherein said malodor control polymer is present in an amount of about 0.01% to about 10% by weight of said composition.

10. The composition of claim 1 wherein said malodor control polymer is a metallated malodor control polymer.

11. The composition of claim 10 wherein said metallated malodor control polymer comprises a metal / polymer weight ratio of 0.001 to 0.01.

12. The composition of claim 10 wherein said metallated malodor control polymer comprises a partially hydrolyzed PVam modified with a C4-C10 alkyl or alkylene.

13. The composition of claim 10 wherein said metallated malodor control polymer comprises a hydrophobic ally modified, 95% hydrolyzed PVam.

14. The composition claim 10 wherein said metallated malodor control polymer comprises a hydrophobic ally modified PEI.

15. The composition of claim 10 wherein said metallated malodor control polymer comprises a C16 to C26 alkyl or alkylene HMP.

16. The composition of claim 10 wherein said metallated malodor control polymer comprises a C16 to C26 alkyl or alkylene HMP.

17. The composition of claim 10 wherein said metal ion is Zn.

18. The malodor control composition of claim 1 wherein said at least one volatile aldehyde has a VP of about 0.001 to about 50 torr .

19. The malodor control composition of claim 1 wherein said at least one volatile aldehyde is selected from the group consisting of 2-ethoxy benzylaldehyde, 2-isopropyl-5-methyl-2-hexenal, 5-methyl furfural, 5-methyl-thiophene-carboxaldehyde, adoxal, p-anisaldehyde, benzylaldehyde, bourgenal, cinnamic aldehyde, cymal, decyl aldehyde, floral super, florhydral, helional, lauric aldehyde, ligustral, lyral, melonal, o-anisaldehyde, pino acetaldehyde, P.T. bucinal, thiophene carboxaldehyde, trans-4-decenal, trans trans 2,4-nonadienal, undecyl aldehyde, and mixtures thereof.

20. The malodor control composition of claim 1 wherein said at least one volatile aldehyde is selected from the group consisting of flor super, o-anisaldehyde, and mixtures thereof.

21. The malodor control composition of claim 1 wherein said at least one volatile aldehydes is present in an amount from about 1% to about 10%, by weight of said perfume mixture.

22. The malodor control composition of claim 1 wherein said at least one volatile aldehyde is present in an amount from about 1% to about 5%, by weight of said perfume mixture, and said acid catalyst is present in an amount of about 0.4% to about 1.5%, by weight of said malodor control composition.

23. The malodor control composition of claim 1 wherein said at least one volatile aldehyde comprises a mixture of volatile aldehydes selected from the group consisting of Accord A, Accord B, Accord C, and mixtures thereof.

24. The malodor control composition of claim 1 wherein said at least one volatile aldehyde comprises a mixture of volatile aldehydes comprising about 1% to about 10% of Accord A, by weight of said perfume mixture.

25. The malodor control composition of claim 1 wherein said acid catalyst has a vapor pressure of about 0.01 to about 2 torr at 25° C.

26. The malodor control composition of claim 1 wherein said acid catalyst is a carboxylic acid.

27. The malodor control composition of claim 1 wherein said acid catalyst is 5-methyl thiophene carboxylic acid.

28. The malodor control composition of claim 1 wherein said acid catalyst is present in an amount from about 0.1% to about 0.4%, by weight of said malodor control composition.

29. The malodor control composition of claim 1 wherein said acid catalyst is present in an amount of about 0.4%, by weight of said malodor control composition.

30. The composition of claim 1 wherein said composition further comprises a buffering agent selected from the group consisting of carboxylic acid, dicarboxcylic acid, N-(2-Acetamido)-2-aminoethanesulfonic acid, and mixtures thereof.

31. The composition of claim 1 wherein said composition further comprises cyclodextrin.

32. The composition of claim 1 wherein said composition comprises a pH of about 6 to about 8.

33. A method of reducing malodor comprising the steps of:
a. providing the composition of claim 1;
b. dispersing an effective amount of said composition on an inanimate surface or in the air.
